(19) United States
(12) Patent Application Publication
(10) Pub. No.: US 2022/0145286 A1
(43) Pub. Date: May 12, 2022

(54) BARCODED CLONAL TRACKING OF GENE TARGETING IN CELLS

(71) Applicants: Chan Zuckerberg Biohub, Inc., San Francisco, CA (US); The Board of Trustees of the Leland Stanford Junior University, Stanford, CA (US)

(72) Inventors: Daniel Dever, Stanford, CA (US); Rajiv Sharma, Stanford, CA (US); Matthew Porteus, Stanford, CA (US); Ravindra Majeti, Stanford, CA (US); Joab Camarena, Stanford, CA (US); Thomas Koehnke, Stanford, CA (US)

(21) Appl. No.: 17/602,843

(22) PCT Filed: Apr. 7, 2020
(86) PCT No.: PCT/US2020/027059

§ 371 Date: Oct. 11, 2021

Related U.S. Application Data
(60) Provisional application No. 62/833,267, filed on Apr. 12, 2019.

(51) Int. Cl. C12N 15/10; C12N 9/22; C12N 15/11; C12N 15/86; C12Q 1/6806

(52) U.S. Cl. CPC C12N 15/1065; C12N 9/22; C12N 15/11; C12N 2750/14143; C12Q 1/6806; C12N 2310/20; C12N 2800/80; C12N 15/86

(57) ABSTRACT

Methods and compositions for monitoring a plurality of independent genomic modifications in cell lineages are provided.

### CROSS-REFERENCE TO RELATED PATENT APPLICATIONS

The present application claims benefit of priority to U.S. Provisional Patent Application No. 62/833,267, filed Apr. 12, 2019, which is incorporated by reference for all purposes.

### SEQUENCE LISTING

The instant application contains a Sequence Listing which has been submitted electronically in ASCII format and is hereby incorporated by reference in its entirety. Said ASCII copy, created on Feb. 29, 2020, is named 103182-1174045_(002510WO)_SL.txt and is 16,824 bytes in size.

### BACKGROUND OF THE INVENTION

Hematopoietic stem cells are a continued source of blood and immune cells. These cells can be useful in a variety of treatments, including e.g., primary immune deficiencies, lysosomal storage disorders, HIV/AIDS, and blood disorders. Gene therapy using integrating retroviral vector, among other delivery mechanisms, have been described. Moreover, targeted gene modification of hematopoietic stem cells using CRISPR/Cas have been described.

### BRIEF SUMMARY OF THE INVENTION

The disclosure provides a method of tracking cell populations comprising an introduced DNA molecule. In some embodiments, the method comprises introducing a plurality of homology recombination donor template polynucleotide sequences into a plurality of cells under conditions such that at least part of the homology recombination donor template polynucleotide sequences are introduced into a target genomic sequence of a cell from the cell population, wherein the homology recombination donor template polynucleotide sequences comprise in the following order: a left homology arm, a coding sequence, and a right homology arm, wherein (1) the coding sequence comprises a silent mutation compared to a wildtype coding sequence of the cell, wherein the plurality of homology recombination donor template polynucleotide sequences comprises different silent mutations and wherein at least two cells receive recombined polynucleotides, each having a different silent mutation; or (2) between the left and right homology arms and outside the coding sequence a barcode sequence is present, wherein the plurality comprises different barcodes and wherein at least two cells receive recombined polynucleotides, each having a different barcode sequence.

In some embodiments, the plurality of homology recombination donor template polynucleotide sequences comprises at least 10 different silent mutations and wherein at least 10, 100, 1000, 10000 or more cells receive recombined polynucleotides, each having a different silent mutation

In some embodiments, the plurality comprises at least 10, 100, 1000, or 10000 different barcodes and wherein at least 10, 100, 1000, or 10000 cells receive recombined polynucleotides, each having a different barcode sequence.

In some embodiments, wherein between the left and right homology arms and outside the coding sequence the barcode sequence is present and wherein following the coding sequences there is a polyA sequence and the barcode is present between the polyA sequence and the right homology arm.

In some embodiments, the cells are primary cells. In some embodiments, the cells are primary hematopoietic cells. In some embodiments, the cells are primary hematopoietic stem cells. In some embodiments, the cells are primary T-cells. In some embodiments, the cells are human cells.

In some embodiments, the introducing comprises providing a targeted nuclease into the cell wherein the targeted nuclease introduces a double-stranded break in the genomic DNA of the cell at a sequence in the genome to which the right and left homology arm sequences have homology. In some embodiments, the targeted nuclease is targeted by a single guide RNA (sgRNA). In some embodiments, the sgRNA comprises one or more modified nucleotides. In some embodiments, the targeted nuclease comprises CRISPR-associated protein (Cas) polypeptide. In some embodiments, the targeted nuclease comprises a zinc finger nuclease (ZFN), a transcription activator-like effector nuclease (TALEN) or a meganuclease.

In some embodiments, the introducing comprises introducing adeno-associated viral (AAV) vectors comprising the homology recombination donor template polynucleotide sequences. In some embodiments, the introducing further comprises introducing into the cells a ribonucleoprotein (RNP) comprising a single guide RNA (sgRNA) and a CRISPR-associated protein (Cas) polypeptide.

In some embodiments, the method further comprises allowing the cell population to divide thereby forming an expanded cell population; and sequencing recombined polynucleotides from the expanded cell population, thereby allowing for tracking of different cells based on the different silent mutations or different barcodes. In some embodiments, the cells are primary hematopoietic cells and the allowing comprises introducing the cells into an animal and the cells divide and optionally differentiate in the animal. In some embodiments, the animal is a human. In some embodiments, the cells are autologous to the animal. In some embodiments, the cells are allogenic to the animal.

In some embodiments, the coding sequence encodes hemoglobin (HBB), Wiskott-Aldrich Syndrome Protein (WAS), Iduronidase (IDUA), Interleukin-7 receptor alpha (Il7RA), Interleukin-2 receptor gamma chain (Il2RG), gp91phox (CYBB), V(D)J recombination-activating protein 1(RAG), V(D)J recombination-activating protein 2 (RAG2), Galactosylceramidase (GALC), Tripeptidyl-peptidase 1(TPP), Glucosylcermidase beta (GBA), Cystic Fibrosis Transmembrane Receptor (CFTR), Forxhead box protein P3 (FOXP3), CD40 Ligand (CD40L), Perforin 1 (PRF1), T-cell Receptor (TCR), Beta-2-microglobulin (B2M), ATP-binding cassette sub-family D member1 (ABCD-1), Brain-derived neurotrophic factor (BDNF), or phenylalanine hydroxylase (PAH).

In some embodiments, the introducing comprises introducing adeno-associated viral (AAV) vectors comprising the homology recombination donor template polynucleotide sequences.

Also provided is a plurality of homology recombination donor template polynucleotide sequences comprising in the following order: a left homology arm, a coding sequence, and a right homology arm, wherein (1) the coding sequence comprises a silent mutation compared to a wildtype coding sequence, wherein the plurality comprises at least two different silent mutations; or (2) between the left and right homology arms and outside the coding sequence a barcode sequence is present, wherein the plurality comprises at least two different barcodes.

In some embodiments, the plurality of homology recombination donor template polynucleotide sequences comprises at least 10, 100, 1000, 10000 or more different homology recombination donor template polynucleotide sequences, each having a different silent mutation. In some embodiments, the plurality comprises at least 10 100, 1000, 10000 or more different homology recombination donor template polynucleotide sequences, each having a different barcode sequence.

In some embodiments, between the left and right homology arms and outside the coding sequence the barcode sequence is present and wherein following the coding sequences there is a polyA sequence and the barcode is present between the polyA sequence and the right homology arm or between the coding sequence and the polyA sequence.

In some embodiments, the coding sequence encodes hemoglobin (HBB), Wiskott-Aldrich Syndrome Protein (WAS), Iduronidase (IDUA), Interleukin-7 receptor alpha (Il7RA), Interleukin-2 receptor gamma chain (Il2RG), gp91phox (CYBB), V(D)J recombination-activating protein 1(RAG), V(D)J recombination-activating protein 2 (RAG2), Galactosylceramidase (GALC), Tripeptidyl-peptidase 1(TPP), Glucosylcermidase beta (GBA), Cystic Fibrosis Transmembrane Receptor (CFTR), Forxhead box protein P3 (FOXP3), CD40 Ligand (CD40L), Perforin 1 (PRF1), T-cell Receptor (TCR), Beta-2-microglobulin (B2M), ATP-binding cassette sub-family D member1 (ABCD-1), Brain-derived neurotrophic factor (BDNF), or phenylalanine hydroxylase (PAH).

In some embodiments, an adeno-associated viral (AAV) vector comprises the homology recombination donor template polynucleotide sequence.

Also provided is a plurality of cells, wherein different cells comprise different homology recombination donor template polynucleotide sequences comprising in the following order: a left homology arm, a coding sequence, and a right homology arm, wherein (1) the coding sequence comprises a silent mutation compared to a wildtype coding sequence, wherein the different homology recombination donor template polynucleotide sequences comprise different silent mutations; or (2) between the left and right homology arms and outside the coding sequence a barcode sequence is present, wherein the different homology recombination donor template polynucleotide sequences comprise different barcodes.

In some embodiments, the plurality of homology recombination donor template polynucleotide sequences comprises at least 10, 100, 1000, 10000 or more different silent mutations and wherein at least 10, 100, 1000, 10000 or more of the cells comprise different homology recombination donor template polynucleotide sequences, each having a different silent mutation.

In some embodiments, the plurality comprises at least 10, 100, 1000, 10000 or more different barcodes and wherein at least 10, 100, 1000, 10000 or more cells comprise different homology recombination donor template polynucleotide sequences, each having a different barcode sequence.

In some embodiments, between the left and right homology arms and outside the coding sequence the barcode sequence is present and wherein following the coding sequences there is a polyA sequence and the barcode is present between the polyA sequence and the right homology arm or between the coding sequence and the polyA sequence.

In some embodiments, the cells are primary cells. In some embodiments, the cells are primary hematopoietic cells. In some embodiments, the cells are primary hematopoietic stem cells. In some embodiments, the cells are primary T-cells. In some embodiments, the cells are human cells.

In some embodiments, the cells comprise a targeted nuclease, wherein the targeted nuclease targets a double-stranded break in the genomic DNA of the cell at a sequence in the genome to which the right and left homology arm sequences have homology. In some embodiments, the targeted nuclease is targeted by a single guide RNA (sgRNA). In some embodiments, the targeted nuclease comprises CRISPR-associated protein (Cas) polypeptide. In some embodiments, the targeted nuclease comprises a zinc finger nuclease (ZFN), a transcription activator-like effector nuclease (TALEN) or a meganuclease. In some embodiments, the adeno-associated viral (AAV) vectors comprise the homology recombination donor template polynucleotide sequences.

In some embodiments, ein the cells further comprise a ribonucleoprotein (RNP) comprising a single guide RNA (sgRNA) and a CRISPR-associated protein (Cas) polypeptide.

In some embodiments, the coding sequence encodes hemoglobin (HBB), Wiskott-Aldrich Syndrome Protein (WAS), Iduronidase (IDUA), Interleukin-7 receptor alpha (Il7RA), Interleukin-2 receptor gamma chain (Il2RG), gp91phox (CYBB), V(D)J recombination-activating protein 1(RAG), V(D)J recombination-activating protein 2 (RAG2), Galactosylceramidase (GALC), Tripeptidyl-peptidase 1(TPP), Glucosylcermidase beta (GBA), Cystic Fibrosis Transmembrane Receptor (CFTR), Forxhead box protein P3 (FOXP3), CD40 Ligand (CD40L), Perforin 1 (PRF1), T-cell Receptor (TCR), Beta-2-microglobulin (B2M), ATP-binding cassette sub-family D member1 (ABCD-1), Brain-derived neurotrophic factor (BDNF), or phenylalanine hydroxylase (PAH).

In some embodiments, the adeno-associated viral (AAV) vectors comprise the homology recombination donor template polynucleotide sequences.

### BRIEF DESCRIPTION OF THE DRAWINGS

FIG. 1. Schematic of Barcode Designs. Left: Diagram of HBB locus in humans in which the first exon in which the most common sickle cell mutation (E6V) is highlighted in red. Donors contain synonymous mutations to introduce sequence diversity without modifying the amino acids produced by the target cells. Right: Schema of donors targeting the AAVS1 locus in which diversity is generated by introducing variable nucleotides following the stop codon of the expression cassette (within the 3′ untranslated region, prior to the poly-adenylation signal). FIG. 1 discloses SEQ ID NOS 13-15, respectively, in order of appearance.

FIG. 2. Edited SCD-CD34⁺ cells. Left: Amplicon-based next generation sequencing from purified rAAV2/6 vector DNA shows highly diverse sequences without substantial overrepresentation of individual barcode clones, resembling a normal distribution of barcodes. The median number of reads mapping to a single barcode (blue) is approximately 6. Right: Schema of 14-day erythroid differentiation protocol from sickle cell disease patient derived CD34+ HSPCs, and HPLC quantitation of hemoglobin species in Mock, non-barcode donor, and barcode donor targeted groups. Equivalent levels of HbA and reduction in HbS were observed with the single sequence and barcoded donors.

FIG. 3. In Vivo Engraftment—Week 6.

FIG. 4. Barcode Analysis Pipeline and Barcodes Shared Between Lineages.

FIG. 5 depicts that highly diverse barcodes were detected in modified CD34+ cells.

FIG. 6 depicts top unique barcodes in vivo maintain HBB reading frame to produce Beta Globin. FIG. 6 discloses SEQ ID NOS 16-22, 21, 23, 21, 24, 21, 25, 21, 26, 21, 27, 21, 28, 21, 29, 21, 30, 21, 31, 21, 32, 21, 33, 21, 34, and 21 respectively, in order of appearance.

FIG. 7a-e: Design and production of barcoded AAV6 donors for long-term genetic tracking of gene targeted cells and their progeny. 7a Schematic of HBB targeting strategy. Top: Unmodified (WT) and barcoded HBB alleles depicted, with location of the E6V (GAG->GTG) sickle cell disease mutation and CRISPR/Cas9 target sites labeled. Bottom: β-globin ORF translation with four barcode pools representing all possible silent mutations encoding amino acids 1-9. FIG. 7a Discloses SEQ ID NOS 35-41, respectively, in order of appearance. 7b Schematic of barcode library generation and experimental design. 7c/d Percentages of reads from each valid barcode identified through amplicon sequencing of plasmids (c) and AAV (d) pools 1, 2, and 4. 7e Recovery of barcodes from untreated genomic DNA containing 1, 3, 10, 30, and 95 individual plasmids containing HBB barcodes. Expected number of barcodes are plotted against the number of barcodes called by the TRACE-seq pipeline after filtering.

FIG. 8a-f Correction of the Sickle Cell Disease-causing E6V mutation using barcoded AAV6 donors in SCD-derived CD34⁺ HSPCs. 8a Experimental design—SCD patient derived CD34⁺ HSPCs edited with CRISPR/Cas9 RNP and electroporation only (mock), single donor (non-BC), or barcode donor (BC) AAV6 HDR templates. 8b SCD correction efficiency (percentage of corrected sickle cell alleles) of non-BC and BC treated groups as a fraction of total NGS reads (e.g. HR reads/[sum of HR reads+unmodified reads].) 8c Representative example of barcode fractions in descending order from one donor at day 14 time point. Right: Top 20 clones represented as stacked bar graph (representing 11.4% of reads). 8d Number of unique barcode alleles comprising the top 50% and top 90% of reads from each treatment condition, sampling approximately 1000 cells per condition. 8e Representative hemoglobin tetramer HPLC chromatograms of RBC differentiated cell lysates at day 14 post treatment. 8f Quantification of total hemoglobin protein expression in each group. Each data point represents an individual biological replicate. HgbA: adult hemoglobin HgF: fetal hemoglobin HbS: sickle hemoglobin. AAV6: Recombinant AAV2/6 vector.

FIG. 9a-f: TRACE-Seq identifies lineage-restricted and multi-potent gene targeted HSPCs in primary NSG transplants. CD34⁺ enriched cord blood-derived HSPCs were cultured in HSPC media containing SCF, FLT3L, TPO, IL-6, and UM-171 for 48 h, electroporated with Cas9 RNP (HBB sgRNA), transduced with AAV6 donors (either BC or non-BC), and cultured for an additional 48 h prior to intrafemoral transplant into sublethally irradiated NSG mice (total manufacturing time was less than 96 hours). 16-18 weeks post transplantation, total BM was collected and analyzed for engraftment by flow cytometry, sorted on lineage markers, and sequenced for unique barcodes. Two independent experiments were performed to assess reproducibility of identifying clonality of gene-targeted HSPCs. 9a Total human engraftment in whole bone marrow, (as measured by proportion of human HLA-ABC⁺ cells). 9b Multilineage engraftment of human CD19⁺, CD33⁺, and HSPCs (CD19⁻CD33⁻CD10⁻CD34⁺). 9c Genome editing efficiency in each indicated sorted human lineage subset as determined by NGS (HR reads/[sum of HR reads+unmodified reads]). 9d Barcodes from each subset were sorted from largest to smallest by percentage of reads. Depicted are the numbers of most abundant, unique barcode alleles comprising the top 50% and top 90% of reads from each lineage of all mice transplanted with BC donor edited HSPCs. Mean±SEM genomes analyzed from each group: CD19⁺: 8500±1000, CD33⁺: 8800±800, HSPC: 1500±500. 9e Correlation between numbers of high confidence barcodes (>0.5%) in lymphoid (grey) and myeloid (black) compartments and total human engraftment (as percent of human and mouse BM-MNCs). Lymphoid and myeloid values plotted for n=9 primary engrafted mice and n=1 secondary engrafted mouse. 9f Correlation between numbers of high confidence barcodes (>0.5%) in lymphoid (grey) and myeloid (black) compartments and HR adjusted engraftment ([human engraftment]×[lineage specific engraftment]×[HR efficiency]). Lymphoid and myeloid values plotted for n=9 primary engrafted mice and n=1 secondary engrafted mouse. 9g Numbers of high confidence barcodes from each mouse which contribute to lymphoid only (CD19⁺), myeloid only (CD33⁺), or both lineages. High confidence barcodes: barcodes with at least 0.5% representation. All points represent individual mice, with the exception of panels e-g (where barcodes from each mouse are separated based on lineage contribution). Error bars depict mean±SEM. p values reflect 2-tailed t-test.

FIG. 10a-b Identification of clonal dynamics of HBB-targeted HSPCs. 10a Top: Experimental schematic. Middle: Flow cytometry plots representing robust bi-lineage engraftment in primary transplant (left, week 18 post-transplant) and secondary transplant (right, week 12 post-transplant). Bottom: Bubble plots representing barcode alleles as unique colors from each indicated sorted population. Shown are the three most abundant clones from all six populations. All other barcodes represented as grey bubbles. 10b Normalized output of barcode alleles with respect to lineage contribution. Total cell output (bar graphs) from indicated barcodes adjusted for both differential lineage output and genome editing efficiency within each subset. Examples of various lineage skewing depicted, with cell counts proportional to the absolute contribution to the xenograft. Skewed output defined as 5-fold or greater bias in absolute cell counts towards lymphoid or myeloid lineages.

FIG. 11a-b: Clonal tracking of AAVS1 barcoded targeted HSPCs in reconstituting primary and secondary NSG transplants. 11a Top: Experimental schematic. Middle: Flow cytometry plots representing bi-lineage engraftment in primary transplant (left, week 18 post-transplant) and secondary transplant (right, week 12 post-transplant). Bottom: Bubble plots representing barcode alleles as unique colors from each indicated sorted population. Shown are the three most abundant clones from all six populations. All other barcodes represented as grey bubbles. 11b Normalized output of barcode alleles with respect to lineage contribution. Total cell output (bar graphs) from indicated barcodes adjusted for both differential lineage output and genome editing efficiency within each subset. Examples of various lineage skewing depicted, with cell counts reflecting relative contributions to the xenograft. One highly engrafted mouse (Mouse 7) depicted of n=5 total. Skewed output defined as 5-fold or greater bias in absolute cell counts towards lymphoid or myeloid lineages.

### DEFINITIONS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “gene” refers to a combination of polynucleotide elements, that when operatively linked in either a native or recombinant manner, provide some product or function. The term “gene” is to be interpreted broadly, and can encompass mRNA, cDNA, cRNA and genomic DNA forms of a gene.

The term “homology-directed repair” or “HDR” refers to a mechanism in cells to accurately and precisely repair double-strand DNA breaks using a homologous template to guide repair. A common form of HDR is homologous recombination (HR).

The term “homologous recombination” or “HR” refers to a genetic process in which nucleotide sequences are exchanged between two similar molecules of DNA. Homologous recombination (HR) is used by cells to accurately repair harmful breaks that occur on both strands of DNA, known as double-strand breaks or other breaks that generate overhanging sequences.

The term “single guide RNA” or “sgRNA” refer to a DNA-targeting RNA containing a guide sequence that targets the Cas nuclease to the target genomic DNA and a scaffold sequence that interacts with the Cas nuclease (e.g., tracrRNA), and optionally, a donor repair template

The term “Cas polypeptide” or “Cas nuclease” refers to a Clustered Regularly Interspaced Short Palindromic Repeats-associated polypeptide or nuclease that cleaves DNA to generate blunt ends at the double-strand break at sites specified by a 20-nucleotide guide sequence contained within a crRNA transcript. A Cas nuclease requires both a crRNA and a tracrRNA for site-specific DNA recognition and cleavage. The crRNA associates, through a region of partial complementarity, with the tracrRNA to guide the Cas nuclease to a region homologous to the crRNA in the target DNA called a “protospacer.”

The term “ribonucleoprotein complex” or “RNP complex” refers to a complex comprising an sgRNA and a Cas polypeptide.

The term “homologous donor adeno-associated viral vector” or “donor adeno-associated viral vector” refers to an adeno-associated viral particle that can express a recombinant donor template for CRISPR-based gene editing via homology-directed repair in a host cell, e.g., primary cell.

The term “recombinant donor template” refers to a nucleic acid stand, e.g., DNA strand that is the recipient strand during homologous recombination strand invasion that is initiated by the damaged DNA, in some cases, resulting from a double-stranded break. The donor polynucleotide serves as template material to direct the repair of the damaged DNA region.

The terms “sequence identity” or “percent identity” in the context of two or more nucleic acids or polypeptides refer to two or more sequences or subsequences that are the same (“identical”) or have a specified percentage of amino acid residues or nucleotides that are identical (“percent identity”) when compared and aligned for maximum correspondence with a second molecule, as measured using a sequence comparison algorithm (e.g., by a BLAST alignment), or alternatively, by visual inspection.

The term “homologous” refers to two or more amino acid sequences when they are derived, naturally or artificially, from a common ancestral protein or amino acid sequence. Similarly, nucleotide sequences are homologous when they are derived, naturally or artificially, from a common ancestral nucleic acid.

The term “primary cell” refers to a cell isolated directly from a multicellular organism. Primary cells typically have undergone very few population doublings and are therefore more representative of the main functional component of the tissue from which they are derived in comparison to continuous (tumor or artificially immortalized) cell lines. In some cases, primary cells are cells that have been isolated and then used immediately. In other cases, primary cells cannot divide indefinitely and thus cannot be cultured for long periods of time in vitro.

The term “gene modified primary cell” or “genome edited primary cell” refers to a primary cell into which a heterologous nucleic acid has been introduced in some cases, into its endogenous genomic DNA.

The term “primary blood cell” refers to a primary cell obtained from blood or a progeny thereof. A primary blood cell can be a stem cell or progenitor cell obtained from blood. For instance, a primary blood cell can be a hematopoietic stem cell or a hematopoietic progenitor cell.

The term “primary immune cell” or “primary leukocyte” refers to a primary white blood cell including but not limited to a lymphocyte, granulocyte, monocyte, macrophage, natural killer cell, neutrophil, basophil, eosinophil, macrophage, stem cell thereof, or progenitor cell thereof. For instance, a primary immune cell can be a hematopoietic stem cell or a hematopoietic progenitor cell. A hematopoietic stem cell or a hematopoietic progenitor cell can give rise to blood cells, including but not limited to, red blood cells, B lymphocytes, T lymphocytes, natural killer cells, neutrophils, basophils, eosinophils, monocytes, macrophages, and all types thereof.

The term “pharmaceutical composition” refers to a composition that is physiologically acceptable and pharmacologically acceptable. In some instances, the composition includes an agent for buffering and preservation in storage, and can include buffers and carriers for appropriate delivery, depending on the route of administration.

The term “pharmaceutical acceptable carrier” refers to a substance that aids the administration of an agent (e.g., Cas nuclease, modified single guide RNA, gene modified primary cell, etc.) to a cell, an organism, or a subject. “Pharmaceutically acceptable carrier” refers to a carrier or excipient that can be included in a composition or formulation and that causes no significant adverse toxicological effect on the patient. Non-limiting examples of pharmaceutically acceptable carrier include water, NaCl, normal saline solutions, lactated Ringer's, normal sucrose, normal glucose, binders, fillers, disintegrants, lubricants, coatings, sweeteners, flavors and colors, and the like. Other pharmaceutical carriers are also useful.

The term “administering or “administration” refers to the process by which agents, compositions, dosage forms and/or combinations disclosed herein are delivered to a subject for treatment or prophylactic purposes. Compositions, dosage forms and/or combinations disclosed herein are administered in accordance with good medical practices taking into account the subject's clinical condition, the site and method of administration, dosage, subject age, sex, body weight, and other factors known to the physician. For example, the terms “administering” or “administration” include providing, giving, dosing and/or prescribing agents, compositions, dosage forms and/or combinations disclosed herein by a clinician or other clinical professional.

The term “treating” refers to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The terms “culture,” “culturing,” “grow,” “growing,” “maintain,” “maintaining,” “expand,” “expanding,” etc., when referring to cell culture itself or the process of culturing, can be used interchangeably to mean that a cell (e.g., primary cell) is maintained outside its normal environment under controlled conditions, e.g., under conditions suitable for survival. In some cases, expansion and/or differentiation can occur in vivo. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

The terms “subject,” “patient,” and “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

Unless defined otherwise, all technical and scientific terms used herein have the same meanings as commonly understood by one of ordinary skill in the art to which this technology belongs. Although exemplary methods, devices and materials are described herein, any methods and materials similar or equivalent to those expressly described herein can be used in the practice or testing of the present technology. For example, the reagents described herein are merely exemplary and that equivalents of such are known in the art. The practice of the present technology can employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR I: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Miller and Calos eds. (1987) Gene Transfer Vectors for Mammalian Cells (Cold Spring Harbor Laboratory); and Makrides ed. (2003) Gene Transfer and Expression in Mammalian Cells (Cold Spring Harbor Laboratory).

### DETAILED DESCRIPTION OF THE INVENTION

### Introduction

The inventors have discovered how to monitor cell expansion and differentiation following targeted genomic modification. Following targeted genomic modification, it can be desirable to follow the progression or progeny cells, especially in situations such as therapies in which a plurality of independently modified cells have been introduced into a patient. When a plurality of cells, each having the same modification event (e.g., when multiple cells are independently modified with the same targeted gene insertion) is introduced into an animal or are cultured, the different cells can expand or differentiate differently. These cells can be especially hard to separately track when they have been modified by a targeted genetic modification method such as CRISPR because of its high accuracy, thereby leaving identical modifications. Thus, one can independently introduce a CRISPR-based mutation into a plurality of cells, for example, and those cells cannot be differentiated by the insertedmutation because the mutations are all identical. However, it is possible secondary mutations can occur in the genome (e.g., due to off-target effects) such that the cells act differently. The present methods address how to monitor different cells having the same targeted modification without knowing additional information about the different cells (e.g., a particular clone of cells might expand more or less due to off-target CRISPR activity, off-target donor integration, random mutations that occur when cells divide, other treatments that the cells or the patient/mouse is exposed to, etc.).

Specifically, the inventors have discovered that one can introduce a barcode sequence into the targeted modification such that independent, otherwise identical, targeted modifications can be monitored by the presence of different barcode sequences. The inventors have discovered several different types of barcoding can be used. In some embodiments, the barcode sequence is introduced as part of an introduced coding sequence as silent mutations. This can be achieved for example in view of the degenerate nature of codons allowing for different nucleotide sequences to encode an identical protein. Alternatively, the same coding sequence can be used but a barcode sequence can be introduced outside the coding sequence as part of the DNA sequence introduced into the target cell.

The methods described herein can involve homology-directed repair in which a double-stranded break is inserted into a target genome site in a cell and the cell's DNA repair mechanisms (homology-directed repair (HDR)) use a donor template DNA as a basis to repair the breakage site. A donor template sequence comprising homology arms flanking a donor sequence will be introduced into the site, allowing for genetic modification at the target site.

The donor template can include two non-overlapping, homologous portions of the target nucleic acid (“homology arms”), wherein the nucleotide sequences are located at the 5′ and 3′ ends (also referred to as “left” and “right” arms) of a nucleotide sequence corresponding to the target nucleic acid to undergo homologous recombination. The donor template can optionally further comprise, inter alia, a coding sequence, a selectable marker, a detectable marker, and/or a cell purification marker.

In some embodiments, the homology arms are the same length. In other embodiments, the homology arms are different lengths. The homology arms can be at least about 10 base pairs (bp), e.g., at least about 10 bp, 15 bp, 20 bp, 25 bp, 30 bp, 35 bp, 45 bp, 55 bp, 65 bp, 75 bp, 85 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or longer. The homology arms can be about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about 1 kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about 1 kb, about 500 bp to about 2 kb, about 500 bp to about 4 kb, about 1 kb to about 2 kb, about 1 kb to about 2 kb, about 1 kb to about 4 kb, or about 2 kb to about 4 kb. The homology arms can be 100% identical across their sequence to the target sequences or some variation can be included (e.g., they can be at least 90, 95, or 99% identical to the target sequence in the cell).

Between the homology arms one or more coding sequence to be introduced into the target site in the genome can be provided. The coding sequence can be any coding sequence desired, including for example, a coding sequence that replaces an endogenous cell coding sequence (for example to replace a defective coding sequence with an functional or more functional coding sequence), that adds a new coding sequence (e.g., a chimeric antigen receptor (CAR) coding sequence), or other coding sequences (including but not limited to marker genes such as green fluorescent protein (GFP). In some embodiments, a hemoglobin coding sequence is introduced, e.g., a coding sequence (e.g., encoding HBB) that is used to replace an allele of hemoglobin associated with sickle cell anemia. In some embodiments, the coding sequence encodes Wiskott-Aldrich Syndrome Protein (WAS), Iduronidase (IDUA), Interleukin-7 receptor alpha (Il7RA), Interleukin-2 receptor gamma chain (Il2RG), gp91phox (CYBB), V(D)J recombination-activating protein 1(RAG), V(D)J recombination-activating protein 2 (RAG2), Galactosylceramidase (GALC), Tripeptidyl-peptidase 1(TPP), Glucosylcermidase beta (GBA), Cystic Fibrosis Transmembrane Receptor (CFTR), Forxhead box protein P3 (FOXP3), CD40 Ligand (CD40L), Perforin 1 (PRF1), T-cell Receptor (TCR), Beta-2-microglobulin (B2M), ATP-binding cassette sub-family D member1 (ABCD-1), Brain-derived neurotrophic factor (BDNF), or phenylalanine hydroxylase (PAH).

In some embodiments, the transgene is a detectable marker or a cell surface marker. In certain instances, the detectable marker is a fluorescent protein such as green fluorescent protein (GFP), enhanced green fluorescent protein (EGFP), red fluorescent protein (RFP), blue fluorescent protein (BFP), cyan fluorescent protein (CFP), yellow fluorescent protein (YFP), mCherry, tdTomato, DsRed-Monomer, DsRed-Express, DSRed-Express2, DsRed2, AsRed2, mStrawberry, mPlum, mRaspberry, HcRed1, E2-Crimson, mOrange, mOrange2, mBanana, ZsYellow1, TagBFP, mTagBFP2, Azurite, EBFP2, mKalama1, Sirius, Sapphire, T-Sapphire, ECFP, Cerulean, SCFP3A, mTurquoise, mTurquoise2, monomeric Midoriishi-Cyan, TagCFP, mTFP1, Emerald, Superfolder GFP, Monomeric Azami Green, TagGFP2, mUKG, mWasabi, Clover, mNeonGreen, Citrine, Venus, SYFP2, TagYFP, Monomeric Kusabira-Orange, mKOk, mKO2, mTangerine, mApple, mRuby, mRuby2, HcRed-Tandem, mKate2, mNeptune, NiFP, mKeima Red, LSS-mKate1, LSS-mKate2, mBeRFP, PA-GFP, PAmCherry1, PATagRFP, TagRFP6457, IFP1.2, iRFP, Kaede (green), Kaede (red), KikGR1 (green), KikGR1 (red), PS-CFP2, mEos2 (green), mEos2 (red), mEos3.2 (green), mEos3.2 (red), PSmOrange, Dronpa, Dendra2, Timer, AmCyan1, or a combination thereof. In other instances, the cell surface marker is a marker not normally expressed on the primary cells such as a truncated nerve growth factor receptor (tNGFR), a truncated epidermal growth factor receptor (tEGFR), CD8, truncated CD8, CD19, truncated CD19, a variant thereof, a fragment thereof, a derivative thereof, or a combination thereof.

The donor template can be used to introduce a precise and specific nucleotide substitution or deletion in a pre-selected gene, or in some cases, a transgene. Any of a number of transcription and translation control elements, including promoter, transcription enhancers, transcription terminators, and the like, may be used in the donor template. In some embodiments, the recombinant donor template of interest includes a promoter. In other embodiments, the recombinant donor template of interest is promoterless. Useful promoters can be derived from viruses, or any organism, e.g., prokaryotic or eukaryotic organisms. Suitable promoters include, but are not limited to, the spleen focus-forming virus promoter (SFFV), elongation factor-1 alpha promoter (EF1α), Ubiquitin C promoter (UbC), phosphoglycerate kinase promoter (PGK), simian virus 40 (SV40) early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc.

In some embodiments, the recombinant donor template further comprises one or more sequences encoding polyadenylation (polyA) signals. Suitable polyA signals include, but are not limited to, SV40 polyA, thymidine kinase (TK) polyA, bovine growth hormone (BGH) polyA, human growth hormone (hGH) polyA, rabbit beta globin (rbGlob) polyA, or a combination thereof. The donor template can also further comprise a non-polyA transcript-stabilizing element (e.g., woodchuck hepatitis virus posttranscriptional regulatory element (WPRE)) or a nuclear export element (e.g., constitutive transport element (CTE)).

Also included between the homology arms in the donor template is a barcode of sufficient complexity to distinguish between other barcodes in a library. In some cases, the number of different barcodes is relatively small (e.g., 2-100 or 5-20) whereas in other embodiments, at least 10², 10³, 10⁴, 10⁵ or more barcodes are used.

In some embodiments the barcodes are composed of silent mutations in the coding sequence such that different donor template sequences are otherwise identical and encode an identical protein, but include different coding sequences due to codon degeneracy such that there are multiple different coding sequences provided (e.g., introduced into a population of cells). The number of possible different coding sequences will be a function of the particular amino acid sequence encoded as well as the number of amino acids encoded. In some embodiments, 2-100 or 5-20 or at least 10², 10³, 10⁴, 10⁵ or more different coding sequences for the same protein are provided in the methods and composition described herein. Thus a library of donor templates can have that many different sequences differing only by the nucleotide sequence that encode the same protein sequence.

Alternatively to the silent mutation barcoding discussed above, or in combination, a separate barcode sequence can also be provided between the homology arms but outside of the coding sequence. The barcode sequence introduced can be included, for example, after a polyA sequence in the donor template such that the barcode does not significantly affect transcription of the coding sequence. In other embodiments, the barcode can be included after the stop codon and before the polyA transcription end signal, and thus can be included in the mRNA transcript.

The barcode sequence can include any number of nucleotides allowing one to distinguish between other donor template molecules. The number of nucleotides required to distinguish will depend on the number of members of the library desired. In some embodiments, the number of nucleotides in the barcode is 2, 3, 4, 5, 6, 7, 8, 9, 10, or more nucleotides. In some embodiments, all of the nucleotides in the barcode are contiguous and in some embodiments, the barcode can be made up or two or more separate discontinuous sequences.

The donor template can be introduced into the target cell in any way desired. For example, the recombinant donor template can be introduced or delivered into a cell via viral gene transfer or electroporation. In some embodiments, the donor template is delivered using an adeno-associated virus (AAV). Any AAV serotype, e.g., human AAV serotype, can be used including, but not limited to, AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), AAV serotype 11 (AAV11), a variant thereof, or a shuffled variant thereof (e.g., a chimeric variant thereof). In some embodiments, an AAV variant has at least 90%, e.g., 90%, 91%, 92%, 93%, 94%, 95%, 96%, 97%, 98%, 99% or more amino acid sequence identity to a wild-type AAV. An AAV1 variant can have at least 90%, e.g., 90%, 91%, 92%, 93%, 94%, 95%, 96%, 97%, 98%, 99% or more amino acid sequence identity to a wild-type AAV1. An AAV2 variant can have at least 90%, e.g., 90%, 91%, 92%, 93%, 94%, 95%, 96%, 97%, 98%, 99% or more amino acid sequence identity to a wild-type AAV2. An AAV3 variant can have at least 90%, e.g., 90%, 91%, 92%, 93%, 94%, 95%, 96%, 97%, 98%, 99% or more amino acid sequence identity to a wild-type AAV3. An AAV4 variant can have at least 90%, e.g., 90%, 91%, 92%, 93%, 94%, 95%, 96%, 97%, 98%, 99% or more amino acid sequence identity to a wild-type AAV4. An AAV5 variant can have at least 90%, e.g., 90%, 91%, 92%, 93%, 94%, 95%, 96%, 97%, 98%, 99% or more amino acid sequence identity to a wild-type AAV5. An AAV6 variant can have at least 90%, e.g., 90%, 91%, 92%, 93%, 94%, 95%, 96%, 97%, 98%, 99% or more amino acid sequence identity to a wild-type AAV6. An AAV7 variant can have at least 90%, e.g., 90%, 91%, 92%, 93%, 94%, 95%, 96%, 97%, 98%, 99% or more amino acid sequence identity to a wild-type AAV7. An AAV8 variant can have at least 90%, e.g., 90%, 91%, 92%, 93%, 94%, 95%, 96%, 97%, 98%, 99% or more amino acid sequence identity to a wild-type AAV8. An AAV9 variant can have at least 90%, e.g., 90%, 91%, 92%, 93%, 94%, 95%, 96%, 97%, 98%, 99% or more amino acid sequence identity to a wild-type AAV9. An AAV10 variant can have at least 90%, e.g., 90%, 91%, 92%, 93%, 94%, 95%, 96%, 97%, 98%, 99% or more amino acid sequence identity to a wild-type AAV10. An AAV11 variant can have at least 90%, e.g., 90%, 91%, 92%, 93%, 94%, 95%, 96%, 97%, 98%, 99% or more amino acid sequence identity to a wild-type AAV11. An AAV12 variant can have at least 90%, e.g., 90%, 91%, 92%, 93%, 94%, 95%, 96%, 97%, 98%, 99% or more amino acid sequence identity to a wild-type AAV12.

In some instances, one or more regions of at least two different AAV serotype viruses are shuffled and reassembled to generate an AAV chimera virus. For example, a chimeric AAV can comprise inverted terminal repeats (ITRs) that are of a heterologous serotype compared to the serotype of the capsid. The resulting chimeric AAV virus can have a different antigenic reactivity or recognition, compared to its parental serotypes. In some embodiments, a chimeric variant of an AAV includes amino acid sequences from 2, 3, 4, 5, or more different AAV serotypes.

Descriptions of AAV variants and methods for generating thereof are found, e.g., in Weitzman and Linden. Chapter 1-Adeno-Associated Virus Biology in Adeno-Associated Virus: Methods and Protocols Methods in Molecular Biology, vol. 807. Snyder and Moullier, eds., Springer, 2011; Potter et al., Molecular Therapy—Methods & Clinical Development, 2014, 1, 14034; Bartel et al., Gene Therapy, 2012, 19, 694-700; Ward and Walsh, Virology, 2009, 386(2):237-248; and Li et al., Mol Ther, 2008, 16(7):1252-1260. AAV virions (e.g., viral vectors or viral particle) described herein can be transduced into primary cells to introduce the recombinant donor template into the cell. A recombinant donor template can be packaged into an AAV viral vector according to any method known to those skilled in the art. Examples of useful methods are described in McClure et al., J Vis Exp, 2001, 57:3378.

As noted above, a DNA nuclease such as an engineered (e.g., programmable or targetable) DNA nuclease can be used to induce genome editing (e.g., by causing a double-stranded break in DNA) of a target nucleic acid sequence. Any suitable DNA nuclease can be used including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof.

In some embodiments, a nucleotide sequence encoding the DNA nuclease is present in a recombinant expression vector and introduced into the target cell(s). In certain instances, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXT1, pSG5, pSVK3, pBPV, pMSG, and pSVLSV40. However, any other vector may be used if it is compatible with the host cell. For example, useful expression vectors containing a nucleotide sequence encoding a Cas9 polypeptide are commercially available from, e.g., Addgene, Life Technologies, Sigma-Aldrich, and Origene.

Depending on the target cell/expression system used, any of a number of transcription and translation control elements, including promoter, transcription enhancers, transcription terminators, and the like, may be used in the expression vector carrying the nuclease coding sequence. Useful promoters can be derived from viruses, or any organism, e.g., prokaryotic or eukaryotic organisms. Suitable promoters include, but are not limited to, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc.

In other embodiments, a nucleotide sequence encoding the DNA nuclease is introduced into a cell as an RNA (e.g., mRNA). The RNA can be produced by any method known to one of ordinary skill in the art. As non-limiting examples, the RNA can be chemically synthesized or in vitro transcribed. In certain embodiments, the RNA comprises an mRNA encoding a Cas nuclease such as a Cas9 polypeptide or a variant thereof. For example, the Cas9 mRNA can be generated through in vitro transcription of a template DNA sequence such as a linearized plasmid containing a Cas9 open reading frame (ORF). The Cas9 ORF can be codon optimized for expression in mammalian systems. In some instances, the Cas9 mRNA encodes a Cas9 polypeptide with an N- and/or C-terminal nuclear localization signal (NLS). In other instances, the Cas9 mRNA encodes a C-terminal HA epitope tag. In yet other instances, the Cas9 mRNA is capped, polyadenylated, and/or modified with 5-methylcytidine. Cas9 mRNA is commercially available from, e.g., TriLink BioTechnologies, Sigma-Aldrich, and Thermo Fisher Scientific.

In yet other embodiments, the DNA nuclease is introduced into a cell as a polypeptide. The polypeptide can be produced by any method known to one of ordinary skill in the art. As non-limiting examples, the polypeptide can be chemically synthesized or in vitro translated. In certain embodiments, the polypeptide comprises a Cas protein such as a Cas9 protein or a variant thereof. For example, the Cas9 protein can be generated through in vitro translation of a Cas9 mRNA described herein. In some instances, the Cas protein such as a Cas9 protein or a variant thereof can be complexed with a single guide RNA (sgRNA) such as a modified sgRNA to form a ribonucleoprotein (RNP). Cas9 protein is commercially available from, e.g., PNA Bio (Thousand Oaks, Calif., USA) and Life Technologies (Carlsbad, Calif., USA).

Crispr/Cas System

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease can require both the crRNA and the tracrRNA for site-specific DNA recognition and cleavage. This system has now been engineered such that the crRNA and tracrRNA can be combined into one molecule (the “single guide RNA” or “sgRNA”), and the crRNA equivalent portion of the single guide RNA can be engineered to guide the Cas (e.g., Cas9) nuclease to target any desired sequence (see, e.g., Jinek et al. (2012) Science 337:816-821; Jinek et al. (2013) eLife 2:e00471; Segal (2013) eLife 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

In some embodiments, the Cas nuclease has DNA cleavage activity. The Cas nuclease can direct cleavage of one or both strands at a location in a target DNA sequence. For example, the Cas nuclease can be a nickase having one or more inactivated catalytic domains that cleaves a single strand of a target DNA sequence.

Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. CRISPR-related endonucleases that are useful are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

“Cas9” refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g., the two catalytic domains are derived from different bacteria species.

Useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of INDEL mutations at off-target DNA sites. Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, the Cas nuclease can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme can contain a mutation at D10, E762, H983 or D986, as well as a mutation at H840 or N863. In some instances, the dCas9 enzyme contains a D10A or D10N mutation. Also, the dCas9 enzyme can include a H840A, H840Y, or H840N. In some embodiments, the dCas9 enzyme comprises D10A and H840A; D10A and H840Y; D10A and H840N; D10N and H840A; D10N and H840Y; or D10N and H840N substitutions. The substitutions can be conservative or non-conservative substitutions to render the Cas9 polypeptide catalytically inactive and able to bind to target DNA.

For genome editing methods, the Cas nuclease can be a Cas9 fusion protein such as a polypeptide comprising the catalytic domain of the type IIS restriction enzyme, FokI, linked to dCas9. The FokI-dCas9 fusion protein (fCas9) can use two guide RNAs to bind to a single strand of target DNA to generate a double-strand break.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) [also referred to as eSpCas9(1.0)], and SpCas9 (K848A/K1003A/R1060A) [also referred to as eSpCas9(1.1)] variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HF1 contains all four mutations).

Zinc Finger Nucleases (ZFNs)

“Zinc finger nucleases” or “ZFNs” are a fusion between the cleavage domain of FokI and a DNA recognition domain containing 3 or more zinc finger motifs. The heterodimerization at a particular position in the DNA of two individual ZFNs in precise orientation and spacing leads to a double-strand break in the DNA. In some cases, ZFNs fuse a cleavage domain to the C-terminus of each zinc finger domain. In order to allow the two cleavage domains to dimerize and cleave DNA, the two individual ZFNs bind opposite strands of DNA with their C-termini at a certain distance apart. In some cases, linker sequences between the zinc finger domain and the cleavage domain requires the 5′ edge of each binding site to be separated by about 5-7 bp. Exemplary ZFNs that are useful include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

ZFNs can generate a double-strand break in a target DNA, resulting in DNA break repair which allows for the introduction of gene modification. DNA break repair can occur via non-homologous end joining (NHEJ) or homology-directed repair (HDR). In HDR, a donor DNA repair template that contains homology arms flanking sites of the target DNA can be provided.

In some embodiments, a ZFN is a zinc finger nickase which can be an engineered ZFN that induces site-specific single-strand DNA breaks or nicks, thus resulting in HDR. Descriptions of zinc finger nickases are found, e.g., in Ramirez et al., Nucl Acids Res, 2012, 40(12):5560-8; Kim et al., Genome Res, 2012, 22(7):1327-33.

TALENs

“TALENs” or “TAL-effector nucleases” are engineered transcription activator-like effector nucleases that contain a central domain of DNA-binding tandem repeats, a nuclear localization signal, and a C-terminal transcriptional activation domain. In some instances, a DNA-binding tandem repeat comprises 33-35 amino acids in length and contains two hypervariable amino acid residues at positions 12 and 13 that can recognize one or more specific DNA base pairs. TALENs can be produced by fusing a TAL effector DNA binding domain to a DNA cleavage domain. For instance, a TALE protein may be fused to a nuclease such as a wild-type or mutated FokI endonuclease or the catalytic domain of FokI. Several mutations to FokI have been made for its use in TALENs, which, for example, improve cleavage specificity or activity. Such TALENs can be engineered to bind any desired DNA sequence.

TALENs can be used to generate gene modifications by creating a double-strand break in a target DNA sequence, which in turn, undergoes NHEJ or HDR. In some cases, a single-stranded donor DNA repair template is provided to promote HDR.

Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55.

Meganucleases

“Meganucleases” are rare-cutting endonucleases or homing endonucleases that can be highly specific, recognizing DNA target sites ranging from at least 12 base pairs in length, e.g., from 12 to 40 base pairs or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric.

In some instances, the meganuclease is naturally-occurring (found in nature) or wild-type, and in other instances, the meganuclease is non-natural, artificial, engineered, synthetic, rationally designed, or man-made. In certain embodiments, the meganuclease includes an I-CreI meganuclease, I-CeuI meganuclease, I-MsoI meganuclease, I-SceI meganuclease, variants thereof, mutants thereof, and derivatives thereof.

Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Natl Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

In some embodiments, the methods comprise introducing into a cell a guide nucleic acid, e.g., DNA-targeting RNA (e.g., a single guide RNA (sgRNA) or a double guide nucleic acid) or a nucleotide sequence encoding the guide nucleic acid (e.g., DNA-targeting RNA). In some embodiments, a modified single guide RNA (sgRNA) comprising a first nucleotide sequence that is complementary to a target nucleic acid and a second nucleotide sequence that interacts with a CRISPR-associated protein (Cas) polypeptide is introduced into a cell, wherein one or more of the nucleotides in the first nucleotide sequence and/or the second nucleotide sequence are modified nucleotides. See, e.g., U.S. Patent Application Publication No. 2019/0032091.

The DNA-targeting RNA (e.g., sgRNA) can comprise a first nucleotide sequence that is complementary to a specific sequence within a target DNA (e.g., a guide sequence) and a second nucleotide sequence comprising a protein-binding sequence that interacts with a DNA nuclease (e.g., Cas9 nuclease) or a variant thereof (e.g., a scaffold sequence or tracrRNA). The guide sequence (“first nucleotide sequence”) of a DNA-targeting RNA can comprise about 10 to about 2000 nucleic acids, for example, about 10 to about 100 nucleic acids, about 10 to about 500 nucleic acids, about 10 to about 1000 nucleic acids, about 10 to about 1500 nucleic acids, about 10 to about 2000 nucleic acids, about 50 to about 100 nucleic acids, about 50 to about 500 nucleic acids, about 50 to about 1000 nucleic acids, about 50 to about 1500 nucleic acids, about 50 to about 2000 nucleic acids, about 100 to about 500 nucleic acids, about 100 to about 1000 nucleic acids, about 100 to about 1500 nucleic acids, about 100 to about 2000 nucleic acids, about 500 to about 1000 nucleic acids, about 500 to about 1500 nucleic acids, about 500 to about 2000 nucleic acids, about 1000 to about 1500 nucleic acids, about 1000 to about 2000 nucleic acids, or about 1500 to about 2000 nucleic acids at the 5′ end that can direct the DNA nuclease (e.g., Cas9 nuclease) to the target DNA site using RNA-DNA complementarity base pairing. In some embodiments, the guide sequence of a DNA-targeting RNA comprises about 100 nucleic acids at the 5′ end that can direct the DNA nuclease (e.g., Cas9 nuclease) to the target DNA site using RNA-DNA complementarity base pairing. In some embodiments, the guide sequence comprises 20 nucleic acids at the 5′ end that can direct the DNA nuclease (e.g., Cas9 nuclease) to the target DNA site using RNA-DNA complementarity base pairing. In other embodiments, the guide sequence comprises less than 20, e.g., 19, 18, 17, 16, 15 or less, nucleic acids that are complementary to the target DNA site. The guide sequence can include 17 nucleic acids that can direct the DNA nuclease (e.g., Cas9 nuclease) to the target DNA site. In some instances, the guide sequence contains about 1 to about 10 nucleic acid mismatches in the complementarity region at the 5′ end of the targeting region. In other instances, the guide sequence contains no mismatches in the complementarity region at the last about 5 to about 12 nucleic acids at the 3′ end of the targeting region.

The protein-binding scaffold sequence (“second nucleotide sequence”) of the DNA-targeting RNA (e.g., sgRNA) can comprise two complementary stretches of nucleotides that hybridize to one another to form a double-stranded RNA duplex (dsRNA duplex). The protein-binding scaffold sequence can be between about 30 nucleic acids to about 200 nucleic acids, e.g., about 40 nucleic acids to about 200 nucleic acids, about 50 nucleic acids to about 200 nucleic acids, about 60 nucleic acids to about 200 nucleic acids, about 70 nucleic acids to about 200 nucleic acids, about 80 nucleic acids to about 200 nucleic acids, about 90 nucleic acids to about 200 nucleic acids, about 100 nucleic acids to about 200 nucleic acids, about 110 nucleic acids to about 200 nucleic acids, about 120 nucleic acids to about 200 nucleic acids, about 130 nucleic acids to about 200 nucleic acids, about 140 nucleic acids to about 200 nucleic acids, about 150 nucleic acids to about 200 nucleic acids, about 160 nucleic acids to about 200 nucleic acids, about 170 nucleic acids to about 200 nucleic acids, about 180 nucleic acids to about 200 nucleic acids, or about 190 nucleic acids to about 200 nucleic acids. In certain aspects, the protein-binding sequence can be between about 30 nucleic acids to about 190 nucleic acids, e.g., about 30 nucleic acids to about 180 nucleic acids, about 30 nucleic acids to about 170 nucleic acids, about 30 nucleic acids to about 160 nucleic acids, about 30 nucleic acids to about 150 nucleic acids, about 30 nucleic acids to about 140 nucleic acids, about 30 nucleic acids to about 130 nucleic acids, about 30 nucleic acids to about 120 nucleic acids, about 30 nucleic acids to about 110 nucleic acids, about 30 nucleic acids to about 100 nucleic acids, about 30 nucleic acids to about 90 nucleic acids, about 30 nucleic acids to about 80 nucleic acids, about 30 nucleic acids to about 70 nucleic acids, about 30 nucleic acids to about 60 nucleic acids, about 30 nucleic acids to about 50 nucleic acids, or about 30 nucleic acids to about 40 nucleic acids.

In some embodiments, the DNA-targeting RNA (e.g., sgRNA) is a truncated form thereof comprising a guide sequence having a shorter region of complementarity to a target DNA sequence (e.g., less than 20 nucleotides in length). In certain instances, the truncated DNA-targeting RNA (e.g., sgRNA) provides improved DNA nuclease (e.g., Cas9 nuclease) specificity by reducing off-target effects. For example, a truncated sgRNA can comprise a guide sequence having 17, 18, or 19 complementary nucleotides to a target DNA sequence (e.g., 17-18, 17-19, or 18-19 complementary nucleotides). See, e.g., Fu et al., Nat. Biotechnol., 32(3): 279-284 (2014).

The DNA-targeting RNA (e.g., sgRNA) can be selected using any of the web-based software described above. As a non-limiting example, considerations for selecting a DNA-targeting RNA can include the PAM sequence for the Cas9 nuclease to be used, and strategies for minimizing off-target modifications. Tools, such as the CRISPR Design Tool, can provide sequences for preparing the DNA-targeting RNA, for assessing target modification efficiency, and/or assessing cleavage at off-target sites.

The DNA-targeting RNA (e.g., sgRNA) can be produced by any method known to one of ordinary skill in the art. In some embodiments, a nucleotide sequence encoding the DNA-targeting RNA is cloned into an expression cassette or an expression vector. In certain embodiments, the nucleotide sequence is produced by PCR and contained in an expression cassette. For instance, the nucleotide sequence encoding the DNA-targeting RNA can be PCR amplified and appended to a promoter sequence, e.g., a U6 RNA polymerase III promoter sequence. In other embodiments, the nucleotide sequence encoding the DNA-targeting RNA is cloned into an expression vector that contains a promoter, e.g., a U6 RNA polymerase III promoter, and a transcriptional control element, enhancer, U6 termination sequence, one or more nuclear localization signals, etc. In some embodiments, the expression vector is multicistronic or bicistronic and can also include a nucleotide sequence encoding a fluorescent protein, an epitope tag and/or an antibiotic resistance marker. In certain instances of the bicistronic expression vector, the first nucleotide sequence encoding, for example, a fluorescent protein, is linked to a second nucleotide sequence encoding, for example, an antibiotic resistance marker using the sequence encoding a self-cleaving peptide, such as a viral 2A peptide. Viral 2A peptides including foot-and-mouth disease virus 2A (F2A); equine rhinitis A virus 2A (E2A); porcine teschovirus-1 2A (P2A) and Thoseaasigna virus 2A (T2A) have high cleavage efficiency such that two proteins can be expressed simultaneously yet separately from the same RNA transcript.

Suitable expression vectors for expressing the DNA-targeting RNA (e.g., sgRNA) are commercially available from Addgene, Sigma-Aldrich, and Life Technologies. The expression vector can be pLQ1651 (Addgene Catalog No. 51024) which includes the fluorescent protein mCherry. Non-limiting examples of other expression vectors include pX330, pSpCas9, pSpCas9n, pSpCas9-2A-Puro, pSpCas9-2A-GFP, pSpCas9n-2A-Puro, the GeneArt® CRISPR Nuclease OFP vector, the GeneArt® CRISPR Nuclease OFP vector, and the like.

In certain embodiments, the DNA-targeting RNA (e.g., sgRNA) is chemically synthesized. DNA-targeting RNAs can be synthesized using 2′-O-thionocarbamate-protected nucleoside phosphoramidites. Methods are described in, e.g., Dellinger et al., J. American Chemical Society 133, 11540-11556 (2011); Threlfall et al., Organic & Biomolecular Chemistry 10, 746-754 (2012); and Dellinger et al., J. American Chemical Society 125, 940-950 (2003).

In particular embodiments, the DNA-targeting RNA (e.g., sgRNA) is chemically modified. As a non-limiting example, the DNA-targeting RNA is a modified sgRNA comprising a first nucleotide sequence complementary to a target nucleic acid (e.g., a guide sequence or crRNA) and a second nucleotide sequence that interacts with a Cas polypeptide (e.g., a scaffold sequence or tracrRNA).

Without being bound by any particular theory, sgRNAs containing one or more chemical modifications can increase the activity, stability, and specificity and/or decrease the toxicity of the modified sgRNA compared to a corresponding unmodified sgRNA when used for CRISPR-based genome editing, e.g., homologous recombination. Non-limiting advantages of modified sgRNAs include greater ease of delivery into target cells, increased stability, increased duration of activity, and reduced toxicity. The modified sgRNAs described herein as part of a CRISPR/Cas9 system provide higher frequencies of on-target genome editing (e.g., homologous recombination), improved activity, and/or specificity compared to their unmodified sequence equivalents.

One or more nucleotides of the guide sequence and/or one or more nucleotides of the scaffold sequence can be a modified nucleotide. For instance, a guide sequence that is about 20 nucleotides in length may have 1 or more, e.g., 1, 2, 3, 4, 5, 6, 7, 8, 9, 10, 11, 12, 13, 14, 15, 16, 17, 18, 19, or 20 modified nucleotides. In some cases, the guide sequence includes at least 2, 3, 4, 5, 6, 7, 8, 9, 10, or more modified nucleotides. In other cases, the guide sequence includes at least 2, 3, 4, 5, 6, 7, 8, 9, 10, 11, 12, 13, 14, 15, 16, 17, 19, 20, or more modified nucleotides. The modified nucleotide can be located at any nucleic acid position of the guide sequence. In other words, the modified nucleotides can be at or near the first and/or last nucleotide of the guide sequence, and/or at any position in between. For example, for a guide sequence that is 20 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, and/or position 20 of the guide sequence. In certain instances, from about 10% to about 30%, e.g., about 10% to about 25%, about 10% to about 20%, about 10% to about 15%, about 15% to about 30%, about 20% to about 30%, or about 25% to about 30% of the guide sequence can comprise modified nucleotides. In other instances, from about 10% to about 30%, e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% of the guide sequence can comprise modified nucleotides.

In certain embodiments, the modified nucleotides are located at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and/or at internal positions within the guide sequence.

In some embodiments, the scaffold sequence of the modified sgRNA contains one or more modified nucleotides. For example, a scaffold sequence that is about 80 nucleotides in length may have 1 or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 76, 77, 78, 79, or 80 modified nucleotides. In some instances, the scaffold sequence includes at least 2, 3, 4, 5, 6, 7, 8, 9, 10, or more modified nucleotides. In other instances, the scaffold sequence includes at least 2, 3, 4, 5, 6, 7, 8, 9, 10, 11, 12, 13, 14, 15, 16, 17, 19, 20, or more modified nucleotides. The modified nucleotides can be located at any nucleic acid position of the scaffold sequence. For example, the modified nucleotides can be at or near the first and/or last nucleotide of the scaffold sequence, and/or at any position in between. For example, for a scaffold sequence that is about 80 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, position 37, position 38, position 39, position 40, position 41, position 42, position 43, position 44, position 45, position 46, position 47, position 48, position 49, position 50, position 51, position 52, position 53, position 54, position 55, position 56, position 57, position 58, position 59, position 60, position 61, position 62, position 63, position 64, position 65, position 66, position 67, position 68, position 69, position 70, position 71, position 72, position 73, position 74, position 75, position 76, position 77, position 78, position 79, and/or position 80 of the sequence. In some instances, from about 1% to about 10%, e.g., about 1% to about 8%, about 1% to about 5%, about 5% to about 10%, or about 3% to about 7% of the scaffold sequence can comprise modified nucleotides. In other instances, from about 1% to about 10%, e.g., about 1%, about 2%, about 3%, about 4%, about 5%, about 6%, about 7%, about 8%, about 9%, or about 10% of the scaffold sequence can comprise modified nucleotides.

In certain embodiments, the modified nucleotides are located at the 3′-end (e.g., the terminal nucleotide at the 3′-end) or near the 3′-end (e.g., within 1, 2, 3, 4, or 5 nucleotides of the 3′-end) of the scaffold sequence and/or at internal positions within the scaffold sequence.

In some embodiments, the modified sgRNA comprises one, two, or three consecutive or non-consecutive modified nucleotides starting at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and one, two, or three consecutive or non-consecutive modified nucleotides starting at the 3′-end (e.g., the terminal nucleotide at the 3′-end) or near the 3′-end (e.g., within 1, 2, 3, 4, or 5 nucleotides of the 3′-end) of the scaffold sequence.

In some instances, the modified sgRNA comprises one modified nucleotide at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and one modified nucleotide at the 3′-end (e.g., the terminal nucleotide at the 3′-end) or near the 3′-end (e.g., within 1, 2, 3, 4, or 5 nucleotides of the 3′-end) of the scaffold sequence.

In other instances, the modified sgRNA comprises two consecutive or non-consecutive modified nucleotides starting at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and two consecutive or non-consecutive modified nucleotides starting at the 3′-end (e.g., the terminal nucleotide at the 3′-end) or near the 3′-end (e.g., within 1, 2, 3, 4, or 5 nucleotides of the 3′-end) of the scaffold sequence.

In yet other instances, the modified sgRNA comprises three consecutive or non-consecutive modified nucleotides starting at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and three consecutive or non-consecutive modified nucleotides starting at the 3′-end (e.g., the terminal nucleotide at the 3′-end) or near the 3′-end (e.g., within 1, 2, 3, 4, or 5 nucleotides of the 3′-end) of the scaffold sequence.

In particular embodiments, the modified sgRNA comprises three consecutive modified nucleotides at the 5′-end of the guide sequence and three consecutive modified nucleotides at the 3′-end of the scaffold sequence.

The modified nucleotides of the sgRNA can include a modification in the ribose (e.g., sugar) group, phosphate group, nucleobase, or any combination thereof. In some embodiments, the modification in the ribose group comprises a modification at the 2′ position of the ribose.

In some embodiments, the modified nucleotide includes a 2′fluoro-arabino nucleic acid, tricycle-DNA (tc-DNA), peptide nucleic acid, cyclohexene nucleic acid (CeNA), locked nucleic acid (LNA), ethylene-bridged nucleic acid (ENA), a phosphodiamidate morpholino, or a combination thereof.

Modified nucleotides or nucleotide analogues can include sugar- and/or backbone-modified ribonucleotides (i.e., include modifications to the phosphate-sugar backbone). For example, the phosphodiester linkages of a native or natural RNA may be modified to include at least one of a nitrogen or sulfur heteroatom. In some backbone-modified ribonucleotides, the phosphoester group connecting to adjacent ribonucleotides may be replaced by a modified group, e.g., a phosphothioate group. In preferred sugar-modified ribonucleotides, the 2′ moiety is a group selected from H, OR, R, halo, SH, SR, NH₂, NHR, NR₂ or ON, wherein R is C₁-C₆ alkyl, alkenyl or alkynyl and halo is F, Cl, Br or I.

In some embodiments, the modified nucleotide contains a sugar modification. Non-limiting examples of sugar modifications include 2′-deoxy-2′-fluoro-oligoribonucleotide (2′-fluoro-2′-deoxycytidine-5′-triphosphate, 2′-fluoro-2′-deoxyuridine-5′-triphosphate), 2′-deoxy-2′-deamine oligoribonucleotide (2′-amino-2′-deoxycytidine-5′-triphosphate, 2′-amino-2′-deoxyuridine-5′-triphosphate), 2′-O-alkyl oligoribonucleotide, 2′-deoxy-2′-C-alkyl oligoribonucleotide (2′-O-methylcytidine-5′-triphosphate, 2′-methyluridine-5′-triphosphate), 2′-C-alkyl oligoribonucleotide, and isomers thereof (2′-aracytidine-5′-triphosphate, 2′-arauridine-5′-triphosphate), azidotriphosphate (2′-azido-2′-deoxycytidine-5′-triphosphate, 2′-azido-2′-deoxyuridine-5′-triphosphate), and combinations thereof.

In some embodiments, the modified sgRNA contains one or more 2′-fluoro, 2′-amino and/or 2′-thio modifications. In some instances, the modification is a 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, 5-amino-allyl-uridine, 5-bromo-uridine, 5-iodo-uridine, 5-methyl-cytidine, ribo-thymidine, 2-aminopurine, 2′-amino-butyryl-pyrene-uridine, 5-fluoro-cytidine, and/or 5-fluoro-uridine.

There are more than 96 naturally occurring nucleoside modifications found on mammalian RNA. See, e.g., Limbach et al., Nucleic Acids Research, 22(12):2183-2196 (1994). The preparation of nucleotides and modified nucleotides and nucleosides are well-known in the art, e.g., from U.S. Pat. Nos. 4,373,071, 4,458,066, 4,500,707, 4,668,777, 4,973,679, 5,047,524, 5,132,418, 5,153,319, 5,262,530, and 5,700,642. Numerous modified nucleosides and modified nucleotides that are suitable for use as described herein are commercially available. The nucleoside can be an analogue of a naturally occurring nucleoside. In some cases, the analogue is dihydrouridine, methyladenosine, methylcytidine, methyluridine, methylpseudouridine, thiouridine, deoxycytodine, and deoxyuridine.

In some cases, the modified sgRNA described herein includes a nucleobase-modified ribonucleotide, i.e., a ribonucleotide containing at least one non-naturally occurring nucleobase instead of a naturally occurring nucleobase. Non-limiting examples of modified nucleobases which can be incorporated into modified nucleosides and modified nucleotides include m5C (5-methylcytidine), m5U (5-methyluridine), m6A (N6-methyladenosine), s2U (2-thiouridine), Um (2′-O-methyluridine), m1A (1-methyl adenosine), m2A (2-methyladenosine), Am (2-1-O-methyl adenosine), ms2m6A (2-methylthio-N6-methyladenosine), i6A (N6-isopentenyl adenosine), ms2i6A (2-methylthio-N6isopentenyladenosine), io6A (N6-(cis-hydroxyisopentenyl) adenosine), ms2io6A (2-methylthio-N6-(cis-hydroxyisopentenyl)adenosine), g6A (N6-glycinylcarbamoyladenosine), t6A (N6-threonyl carbamoyladenosine), ms2t6A (2-methylthio-N6-threonyl carbamoyladenosine), m6t6A (N6-methyl-N6-threonylcarbamoyladenosine), hn6A (N6.-hydroxynorvalylcarbamoyl adenosine), ms2hn6A (2-methylthio-N6-hydroxynorvalyl carbamoyladenosine), Ar(p) (2′-O-ribosyladenosine(phosphate)), I (inosine), m11 (1-methylinosine), m′Im (1,2′-O-dimethylinosine), m3C (3-methylcytidine), Cm (2T-O-methylcytidine), s2C (2-thiocytidine), ac4C (N4-acetylcytidine), f5C (5-fonnylcytidine), m5Cm (5,2-O-dimethylcytidine), ac4Cm (N4acetyl2TOmethylcytidine), k2C (lysidine), m1G (1-methylguanosine), m2G (N2-methylguanosine), m7G (7-methylguanosine), Gm (2′-O-methylguanosine), m22G (N2,N2-dimethylguanosine), m2Gm (N2,2′-O-dimethylguanosine), m22Gm (N2,N2,2′-O-trimethylguanosine), Gr(p) (2′-O-ribosylguanosine(phosphate)), yW (wybutosine), o2yW (peroxywybutosine), OHyW (hydroxywybutosine), OHyW* (undermodified hydroxywybutosine), imG (wyosine), mimG (methylguanosine), Q (queuosine), oQ (epoxyqueuosine), galQ (galtactosyl-queuosine), manQ (mannosyl-queuosine), preQo (7-cyano-7-deazaguanosine), preQi (7-aminomethyl-7-deazaguanosine), G (archaeosine), D (dihydrouridine), m5Um (5,2′-O-dimethyluridine), s4U (4-thiouridine), m5s2U (5-methyl-2-thiouridine), s2Um (2-thio-2′-O-methyluridine), acp3U (3-(3-amino-3-carboxypropyl)uridine), ho5U (5-hydroxyuridine), mo5U (5-methoxyuridine), cmo5U (uridine 5-oxyacetic acid), mcmo5U (uridine 5-oxyacetic acid methyl ester), chm5U (5-(carboxyhydroxymethyl)uridine)), mchm5U (5-(carboxyhydroxymethyl)uridine methyl ester), mcm5U (5-methoxycarbonyl methyluridine), mcm5Um (S-methoxycarbonylmethyl-2-O-methyluridine), mcm5s2U (5-methoxycarbonylmethyl-2-thiouridine), nm5s2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyl uridine), ncm5Um (5-carbamoylmethyl-2′-O-methyluridine), cmnm5U (5-carboxymethylaminomethyluridine), cnmm5Um (5-carboxymethylaminomethyl-2-L-Omethyluridine), cmnm5s2U (5-carboxymethylaminomethyl-2-thiouridine), m62A (N6,N6-dimethyladenosine), Tm (2′-O-methylinosine), m4C (N4-methylcytidine), m4Cm (N4,2-O-dimethylcytidine), hm5C (5-hydroxymethylcytidine), m3U (3-methyluridine), cm5U (5-carboxymethyluridine), m6Am (N6,T-O-dimethyladenosine), rn62Am (N6,N6,O-2-trimethyladenosine), m2′7G (N2,7-dimethylguanosine), m2′2′7G (N2,N2,7-trimethylguanosine), m3Um (3,2T-O-dimethyluridine), m5D (5-methyldihydrouridine), f5Cm (5-formyl-2′-O-methylcytidine), m1Gm (1,2′-O-dimethylguanosine), m′Am (1,2-O-dimethyl adenosine)irinomethyluridine), tm5s2U (S-taurinomethyl-2-thiouridine)), imG-14 (4-demethyl guanosine), imG2 (isoguanosine), or ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, and combinations thereof.

In some embodiments, the phosphate backbone of the modified sgRNA is altered. The modified sgRNA can include one or more phosphorothioate, phosphoramidate (e.g., N3′-P5′-phosphoramidate (NP)), 2′-O-methoxy-ethyl (2′MOE), 2′-O-methyl-ethyl (2′ME), and/or methylphosphonate linkages. In certain instances, the phosphate group is changed to a phosphothioate, 2′-O-methoxy-ethyl (2′MOE), 2′-O-methyl-ethyl (2′ME), N3′-P5′-phosphoramidate (NP), and the like.

In particular embodiments, the modified nucleotide comprises a 2′-O-methyl nucleotide (M), a 2′-O-methyl, 3′-phosphorothioate nucleotide (MS), a 2′-O-methyl, 3′thioPACE nucleotide (MSP), or a combination thereof.

In some instances, the modified sgRNA includes one or more MS nucleotides. In other instances, the modified sgRNA includes one or more MSP nucleotides. In yet other instances, the modified sgRNA includes one or more MS nucleotides and one or more MSP nucleotides. In further instances, the modified sgRNA does not include M nucleotides. In certain instances, the modified sgRNA includes one or more MS nucleotides and/or one or more MSP nucleotides, and further includes one or more M nucleotides. In certain other instances, MS nucleotides and/or MSP nucleotides are the only modified nucleotides present in the modified sgRNA.

It should be noted that any of the modifications described herein may be combined and incorporated in the guide sequence and/or the scaffold sequence of the modified sgRNA.

In some cases, the modified sgRNAs also include a structural modification such as a stem loop, e.g., M2 stem loop or tetraloop.

The chemically modified sgRNAs can be used with any CRISPR-associated or RNA-guided technology. As described herein, the modified sgRNAs can serve as a guide for any Cas9 polypeptide or variant thereof, including any engineered or man-made Cas9 polypeptide. The modified sgRNAs can target DNA and/or RNA molecules in isolated cells or in vivo (e.g., in an animal).

A library (e.g., a plurality) of donor template polynucleotides as described herein (e.g., comprising different barcodes and the same coding sequence, optionally with the barcode being part of the coding sequence) can be introduced into any type of cells as desired. The methods can be used to monitor development of a cell population, in vitro or in vivo. In some embodiments, the cells are primary cells, or expanded from primary cells from an animal. The cells can be genetically altered as described herein and then reintroduced into the animal (e.g., the cells would in this case be autologous) and monitored for development by monitoring the barcodes present in the resulting cell population. Alternatively the cells can be allogenic, i.e., obtained from a first animal, genetically modified and then introduced into a second animal (generally of the same species and optionally matched for MHC/HLA). In some embodiments, one can determine the relative number of cells having different barcodes and/or determine whether different barcodes result in different cell lineages. This is useful for example for identifying potential cancer risks, for example in situations in which one of the cells contains secondary mutations resulting in uncontrolled division compared to the other introduced cells or develop into different lineages than remaining introduced cells. This method is also useful for tracking normal regenerative cell development from gene-targeted stem and progenitor cells in vivo. Thus, this method can be applicable for understanding the fundamentals of gene targeting in stem cells and information gained from this knowledge can be parlayed into advancing gene targeting methodologies.

A library of donor template polynucleotides as described herein (e.g., comprising different barcodes and the same coding sequence, optionally with the barcode being part of the coding sequence or separate from the coding sequence) can have, for example, 2-100 or 5-20 members, or in some embodiments, at least 10², 10³, 10⁴, 10⁵ or more members (e.g., 10²-10⁵) members. Moreover, the library of polynucleotides introduced into cells will generate a library of cells. Accordingly, also provided is a plurality of cells where 2-100 or 5-20 cells, or in some embodiments, at least 10², 10³, 10⁴, 10⁵ or more cells (e.g., 10²-10⁵) cells, wherein each cell comprises a different donor template polynucleotide as described herein (e.g., comprising different barcodes and the same coding sequence, optionally with the barcode being part of the coding sequence or separate from the coding sequence). The cells can be any cells for example describe herein.

Genome nucleotide sequencing can be used to determine the barcode sequence in each cell in a lineage or following cell division. The quantity of different barcode sequences will indicate the relative accumulation of cells having different donor template polynucleotides. As noted above while the genomic target sequence in the cells may be altered in an identical manner between cells (aside from the barcode) the cells will be the result of independent editing events and as such may different for example in off-target genomic effects and thus accumulation of progeny of different altered cells may differ. For example if an off-target effect resulted in oncogenic activity.

Any type of genomic nucleotide sequencing can be used. DNA sequencing techniques include dideoxysequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like. These sequencing approaches can thus be used to sequence target nucleic acids of interest, for example the barcode region and one or more non-variable region directly flanking the barcode.

Certain high-throughput methods of sequencing comprise a step in which individual molecules are spatially isolated on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al., Science, 327: 78-81 (2010)), arrays of wells, which may include bead- or particle-bound templates (such as with 454, e.g. Margulies et al, Nature, 437: 376-380 (2005) or Ion Torrent sequencing, U.S. Patent Publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kimet al, Science, 316: 1481-1414 (2007)). Such methods may comprise amplifying the isolated molecules either before or after they are spatially isolated on a solid surface. Prior amplification may comprise emulsion-based amplification, such as emulsion PCR, or rolling circle amplification. In some embodiments, sequencing is performed on the Illumina™ MiSeqplatform, which uses reversible-terminator sequencing by synthesis technology (see, e.g., Shen et al. (2012) BMC Bioinformatics 13:160; Junemann et al. (2013) Nat. Biotechnol. 31(4):294-296; Glenn (2011) Mol. Ecol. Resour. 11(5):759-769; Thudi et al. (2012) Brief Funct. Genomics 11(1):3-11).

In some embodiments, data is obtained in the form of paired end (for example, 150 bp) reads derived from next generation sequencing of the barcoded region of the genome. This region contains the editing or CRISPR cut site, barcode region with variable base pairs, and “anchor bases” which are the two base pairs flanking the CRISPR cut site that are always modified following homologous recombination (HR). In some embodiments, paired end reads are merged together, for example, using the PEAR tool (J. Zhang, et al., Bioinformatics. 2014 Mar. 1; 30(5):614-20). Merged reads are then aligned to a master read sequence, for example, using a Smith-Waterman alignment algorithm. In some embodiments, reads with low quality alignment are excluded from further analysis. In some embodiments, correctly aligned reads are then binned into three categories: wildtype unmodified reads, reads derived from Non-Homologous End Joining (NHEJ) alleles, and reads derived from correctly modified HR alleles. Reads are determined to be derived from NHEJ alleles if they contain any insertions or deletions in the region flanking the CRISPR cut site. WT reads are non-NHEJ reads without anchor base modification. HR reads are reads without insertions or deletions with correctly modified anchor base sequences. In some embodiments, the rest of the barcode analysis is performed exclusively with the HR reads that contain the modified barcoded DNA sequence. In some embodiments, the HR reads are analyzed using a modified version of the TUBA-seq pipeline (Rogers, Z. N., et al., Nat Methods. 2017 July; 14(7):737-742). TUBA-seq trains a sequencing error model, and clusters barcodes based on that model using the DADA2 clustering algorithm (Callahan B J, et al., Nat Methods. 2016; 13(7):581-3). More specifically, two regions are extracted from each read: the barcode region, and the non-variable regions directly flanking the barcode. In some embodiments, an error model is trained using the non-variable sequences. Then, barcode sequences are clustered into read groups using the derived error model. The sequence and size associated with each read group is output as the sequence and number of each barcode in the original read set. These values and sequences are used for subsequent analysis.

### EXAMPLE

### Example 1

### Methods

### Cloning ITR-Containing Barcoded Plasmid Libraries

To generate ITR-containing plasmids with barcoded homologous donor templates, degenerate nucleotides were first incorporated into gBlocks (IDT) or oligonucleotides for PCR to introduce variable nucleotides into the coding sequence (HBB Donor) or 3′ UTR (AAVS1 Donors). Libraries of plasmids were transformed into XL1-Blue chemically-competent (Cat. 200249, Agilent) or NEB 10-beta electrocompetent E. coli (C3020K, New England Biolabs) and grown for 14-16 hours. Colonies (with 1 colony that should contain 1 barcoded sequence) were pooled in LB-Amp media, and enough colonies were pooled together to obtain the theoretical maximum number of barcodes for a particular library based on the diversity calculated. Then, pooled barcoded ITR-AAV library plasmid DNA was extracted using a ZymoPURE II Maxiprep kit (D4202, Zymo Research). This plasmid DNA was then used to make AAV6 homologous donor templates are described below.

### Producing and Purifying Barcoded AAV6 Donor Templates

Briefly, 293T cells seeded the previous day at 1.1×10⁷ cells per 15-cm dish were transfected using polyethylenimine (PEI) with 6 mg of pAAV-MCS plasmid containing the donor along with 22 mg of pDGM6 (kindly provided by D. Russell). Cells were then lysed by three freeze-thaw cycles and treated with Benzonase, and rAAV6 particles were purified by iodixanol density gradient centrifugation. Extracted rAAV6 was then exchanged in PBS with 5% sorbitol using either a 1×10⁴ molecular weight cut off (MWCO) Slide-ALyzer G2 dialysis Cassette (Thermo Fisher Scientific) or an Amicon centrifugal filter 1×10⁵ MWCO (Millipore Sigma) following the manufacturer's instructions. Titers were measured after buffer exchange as described previously. Alternatively, AAV6 was produced and purified by Vigene Biosciences according to their protocols.

### Barcode Sequencing Analysis

Data was obtained in the form of paired end 150 bp reads derived from next generation sequencing of the barcoded region of the genome. This region contains the editing or CRISPR cut site, barcode region with variable basepairs, and “anchor bases” which are the two basepairs flanking the CRISPR cut site that are always modified following homologous recombination (HR).

Paired end reads were merged together using the PEAR tool. Merged reads were then aligned to a master read sequence using a Smith-Waterman alignment algorithm. Reads with low quality alignment are excluded from further analysis. Correctly aligned reads were then binned into three categories: wildtype unmodified reads, reads derived from Non-Homologous End Joining (NHEJ) alleles, and reads derived from correctly modified HR alleles. Reads were determined to be derived from NHEJ alleles if they contain any insertions or deletions in the region flanking the CRISPR cut site. WT reads are non-NHEJ reads without anchor base modification. HR reads are reads without insertions or deletions with correctly modified anchor base sequences. The rest of the barcode analysis is performed exclusively with the HR reads which contain the modified barcoded DNA sequence.

The HR reads were analyzed using a modified version of the TUBA-seq pipeline. TUBA-seq trains a sequencing error model, and clusters barcodes based on that model using the DADA2 clustering algorithm. More specifically, two regions are extracted from each read: the barcode region, and the non-variable regions directly flanking the barcode. An error model was trained using the non-variable sequences. Then, barcode sequences are clustered into read groups using the derived error model. The sequence and size associated with each read group is output as the sequence and number of each barcode in the original read set. These values and sequences are used for subsequent analysis.

### Results

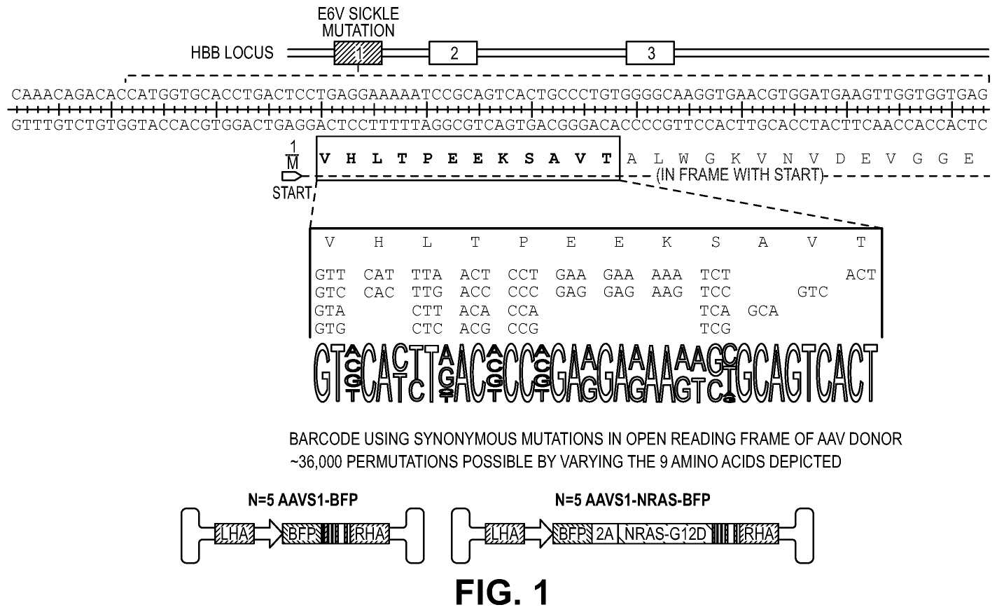

After designing AAV transfer plasmid donors as illustrated in FIG. 1 (see Methods), we performed Sanger sequencing of multiple colonies from the donor libraries confirmed efficient generation of the expected donor constructs containing many differences within the expected variable regions. Amplicon sequencing of rAAV2/6 genomic DNA produced using the pooled plasmid libraries confirmed highly diverse libraries for all donors produced. Importantly, libraries did not exhibit overrepresentation of any barcode sequences (FIG. 1, left).

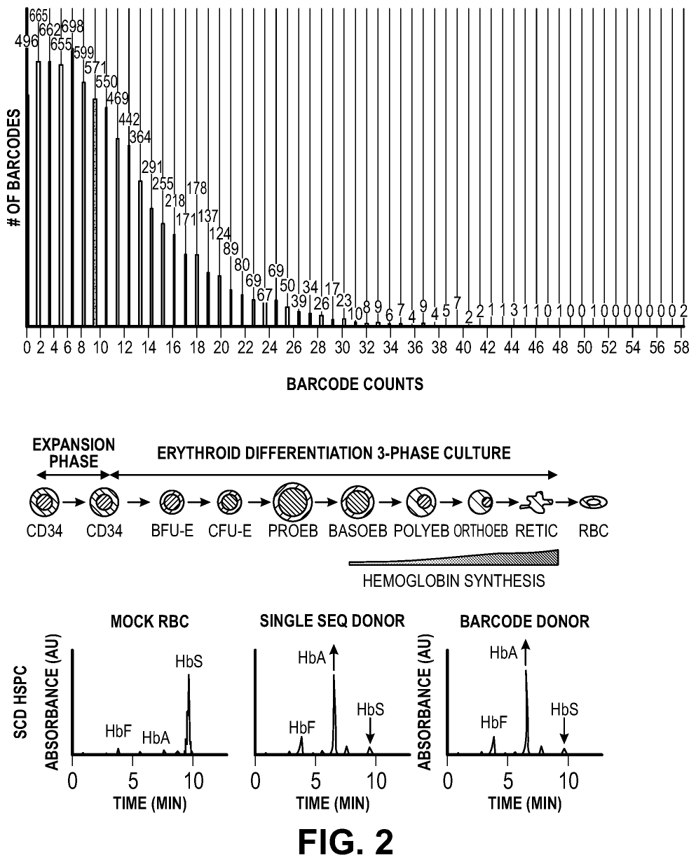

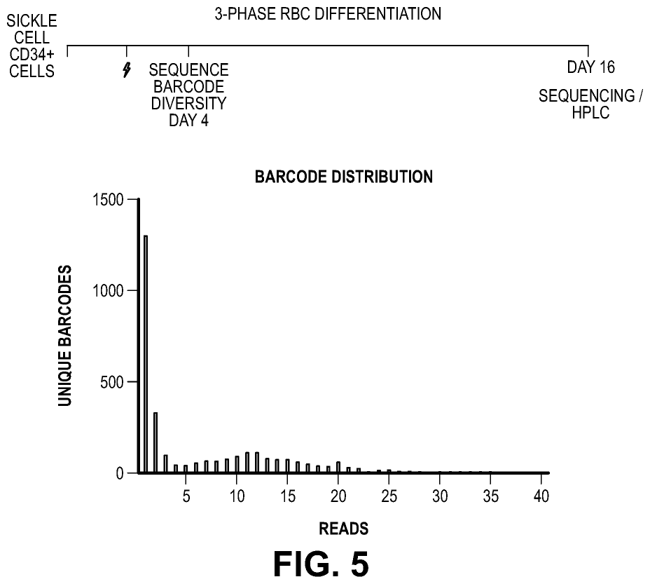

Barcoded rAAV6 donors were capable of supporting genome editing of the HBB locus of sickle cell patient derived, CD34+ HSPCs with efficiencies that were not statistically significantly different from the previously characterized non-barcoded donor. Following a 14-day erythroid differentiation protocol, these edited cells were able to produce hemoglobin levels comparable to the non-barcoded donor (FIG. 2, right). Importantly, the barcoded HBB-targeted HSPCs contained hundreds to thousands of unique barcodes. This highlights that the correction from sickle hemoglobin to adult hemoglobin is representative from a population of cells that are very diverse in their hemoglobin barcoded signatures but similar in the hemoglobin output (FIG. 5).

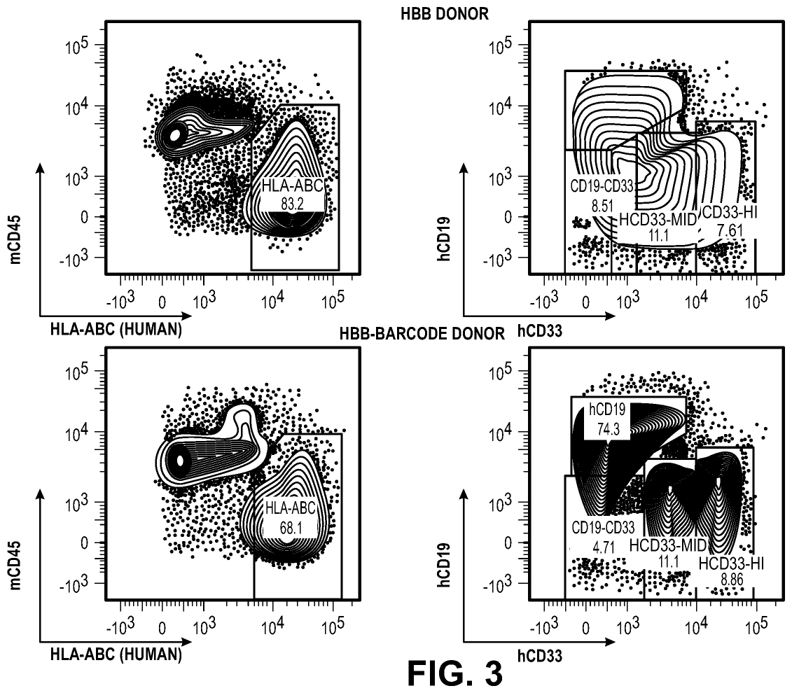

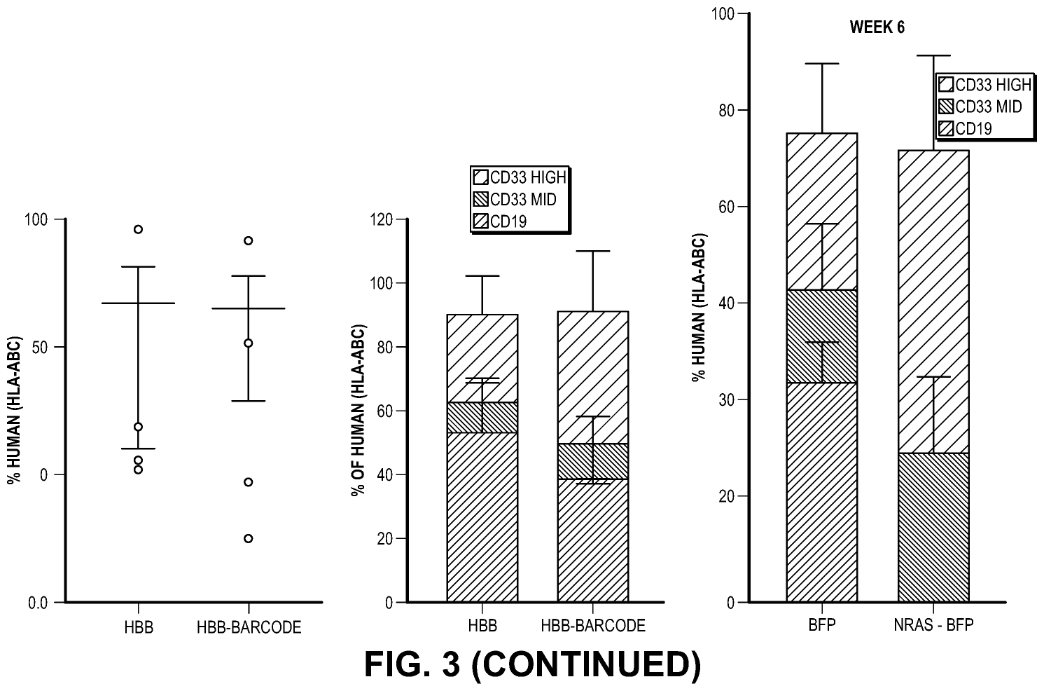

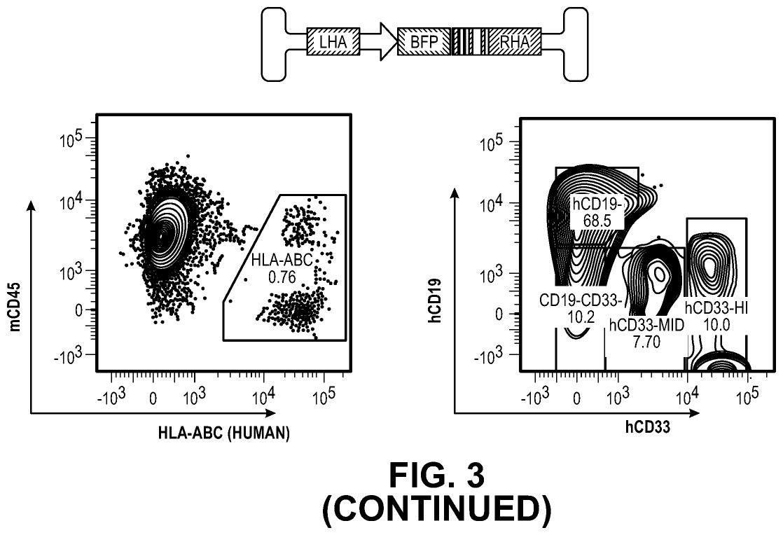

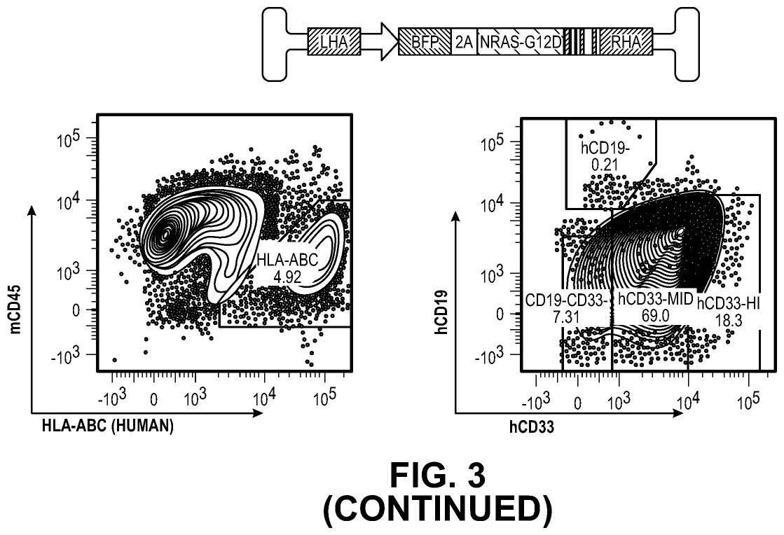

Next, we asked whether barcode gene targeted HSPCs were capable of robust reconstitution of multiple blood lineages in a mouse xenograft model. Cord blood CD34+ cells were targeted as previously described and 2×10⁵ cells were transplanted intra-femorally into sub-lethally irradiated NSG mice (age 6-8 weeks). Using reagents targeting the HBB locus, cells edited using barcoded and non-barcoded donors exhibited similar levels of human engraftment and supported bilineage (CD33+ myeloid and CD19+ lymphoid) engraftment (FIG. 3, left). Using donors which target the AAVS1 locus with a BFP expression cassette, we observed similar bilineage engraftment within 6 weeks of transplantation (FIG. 3, upper right), while the BFP+NRAS^{mut} cassette resulted in a myeloid skewed engraftment lacking substantial levels of CD19+ output within the mice.

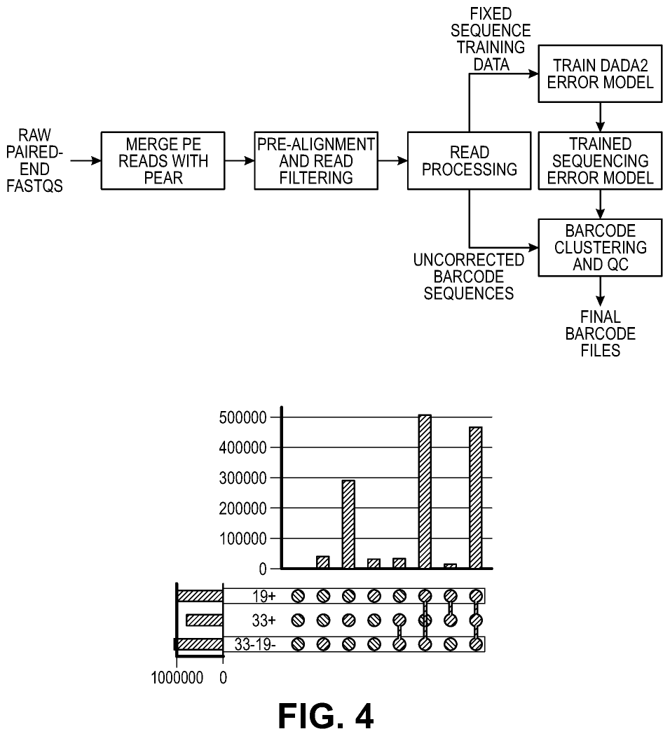

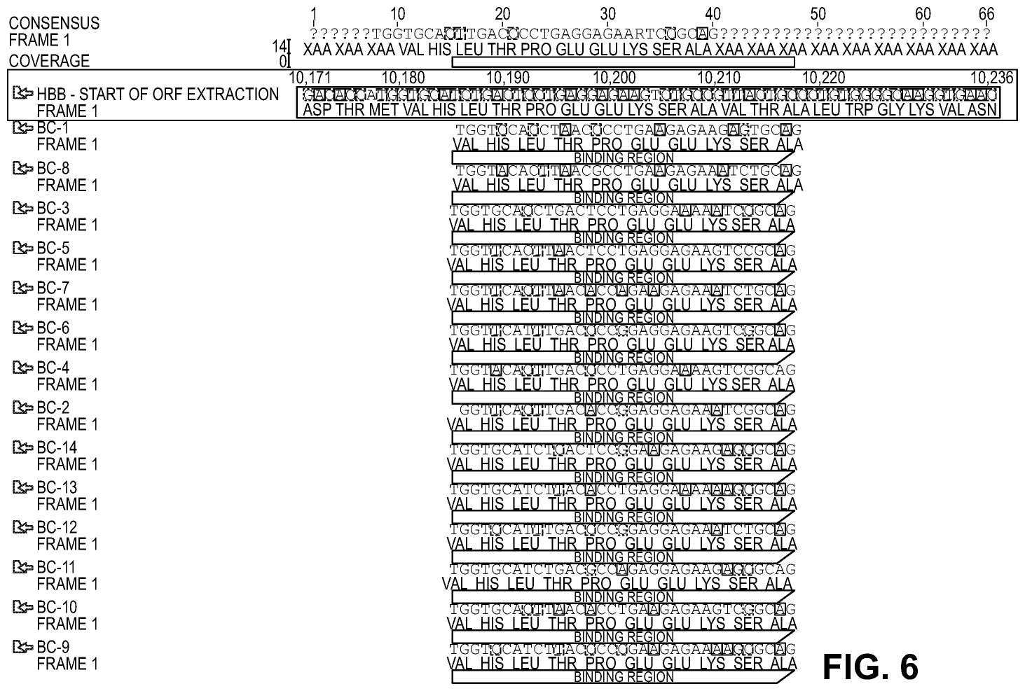

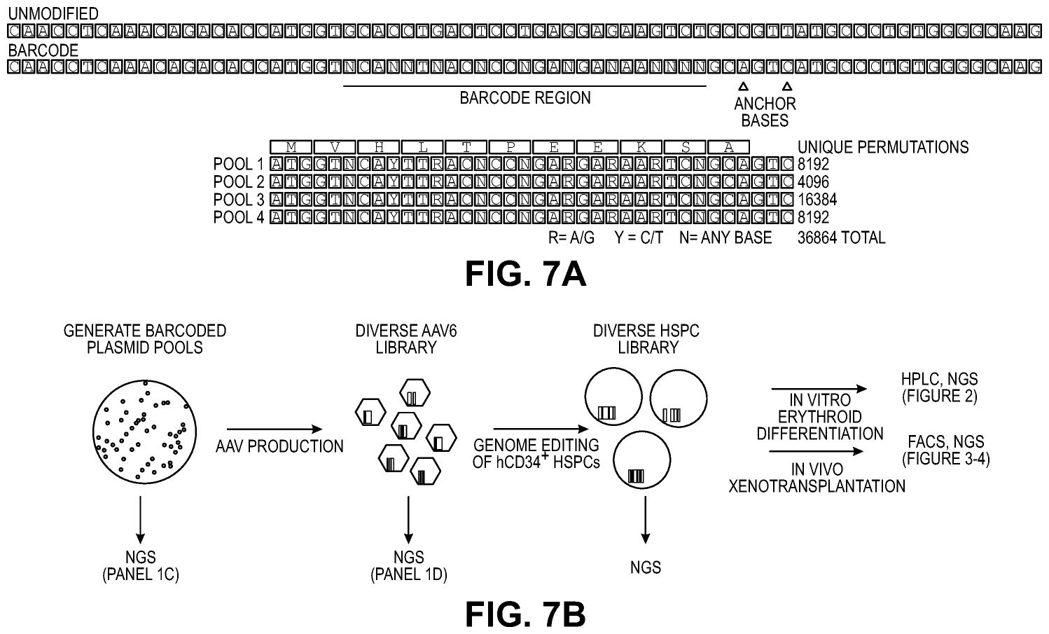

We performed femoral aspirates at weeks 6 and 12 and sacrificed the mice 18 weeks post engraftment. At each timepoint, bone marrow cells were stained for FACS sorting and sorted on Human, CD19, CD33-High, and CD33-Mid gates as well as GPA+ and HSPC gates upon sacrifice. Genomic DNA was isolated from each sorted fraction and the barcode regions were amplified for high throughput sequencing and subsequent analysis (see Methods). In preliminary analyses, we observed hundreds of barcodes both shared between hematopoietic lineages and unique to myeloid and lymphoid lineages (FIG. 4, Right). These data show that HBB and AAVS1 barcoded targeted HSPCs are able to engraft long-term and produce differentiated lineages comparable to non-barcoded targeted HSPCs. These data also show that our gene targeting methodologies are effective in multi-lineage potent stem and progenitor cells. As expected, all of the top HBB barcodes identified from the sorted cells, still maintained the correct coding sequence for hemoglobin (FIG. 6).

### Example 2

The following example provides further details from the experiments discussed above as well as additional information.

Targeted DNA correction of disease-causing mutations in hematopoietic stem and progenitor cells (HSPCs) may usher in a new class of medicines to treat genetic diseases of the blood and immune system. With state-of-the-art methodologies, it is now possible to correct disease-causing mutations at high frequencies in HSPCs by combining ribonucleoprotein (RNP) delivery of Cas9 and chemically modified sgRNAs with homologous DNA donors via recombinant adeno-associated viral vector serotype six (AAV6). However, because of the precise nucleotide-resolution nature of gene correction, these current approaches do not allow for clonal tracking of gene targeted HSPCs. Here, we describe Tracking Recombination Alleles in Clonal Engraftment using sequencing (TRACE-Seq), a novel methodology that utilizes barcoded AAV6 donor template libraries, carrying either in-frame silent mutations or semi-randomized nucleotide sequences outside the coding region, to track the in vivo lineage contribution of gene targeted HSPC clones. By targeting the HBB gene with an AAV6 donor template library consisting of 20,000 possible unique exon 1 in-frame silent mutations, we track the hematopoietic reconstitution of HBB targeted myeloid-skewed, lymphoid-skewed, and balanced multi-lineage repopulating human HSPC clones in immunodeficient mice. We anticipate that this methodology has the potential to be used for HSPC clonal tracking of Cas9 RNP and AAV6-mediated gene targeting outcomes in translational and basic research settings.

### Introduction

Genetic diseases of the blood and immune system, including the hemoglobinopathies and primary immunodeficiencies, affect millions of people worldwide with limited treatment options. Clinical development of ex vivo lentiviral (LV)-mediated gene addition in hematopoietic stem and progenitor cells (HSPCs) has demonstrated that a patient's own HSPCs can be modified and re-transplanted to restore proper cell function in the hematopoietic system [High, K. A. & Roncarolo, M. G. Gene Therapy. N Engl J Med 381, 455-464 (2019)]. While no severe adverse events have been reported resulting from insertional mutagenesis in more than 200 patients transplanted with LV ex vivo manipulated HSPCs [Cavazzana, M. et al., Nat Rev Drug Discov 18, 447-462 (2019)], efficacy in restoring protein/cell function and ultimately disease amelioration has varied. In some diseases, this lack of therapeutic efficacy is possibly the result of irregular spatiotemporal transgene expression due to the semi-random integration patterns of LVs.

Tracking the transgene integration sites (IS) by deep sequencing has been used to “barcode” clones in heterogeneous cell populations that contribute to blood reconstitution in the human transplantation setting. In clinical trials, IS methodology has been used to track genetically modified memory T-cells [Biasco, L. et al., Sci Transl Med 7, 273ra213 (2015)], waves of hematopoietic repopulation kinetics [Biasco, L. et al., Cell Stem Cell 19, 107-119 (2016)], as well as dynamics and outputs of HSPC subpopulations in autologous graft composition [Scala, S. et al., Nat Med 24, 1683-1690 (2018)]. These seminal studies provided new insights into the reconstitution of human hematopoiesis following autologous transplantation. Importantly, IS can also provide evidence of potential concerning integration patterns in tumor-suppressor genes, like PTEN [Mamcarz, E. et al., N Engl J Med 380, 1525-1534 (2019)], TET2 [Fraietta, J. A. et al., Nature 558, 307-312 (2018)] and NF1 [Marktel, S. et al., Nat Med 25, 234-241 (2019)], which can be closely monitored during long-term follow-up to predict future severe adverse events.

Genetic barcoding on the DNA level has been used to track the in vitro [Porter, S. N. et al., Genome Biol 15, R75 (2014)] and in vivo [Lu, R. et al., Nature biotechnology 29, 928-933 (2011); Yabe, I. M. et al., Mol Ther Methods Clin Dev 11, 143-154 (2018); Wu, C. et al., Cell Stem Cell 14, 486-499 (2014); Kristiansen, T. A. et al., Immunity 45, 346-357 (2016)] clonal dynamics of heterogeneous mammalian cellular populations and offers several advantages over lentiviral IS tracking, although it has not been used clinically. First, the amplified region is known and nearly the same for each barcode simplifying recovery from targeted cells, as opposed to semi-random LV integrations, which require amplification of unknown sequences. Second, it is far less likely for differences in amplification efficiency or secondary structure to lead to drop off or mis-quantification of clone sizes [Thielecke, L. et al., Sci Rep 7, 43249 (2017)]. Altogether, genetic barcoding, combined with high-throughput sequencing, can enable sensitive and quantitative assessment of heterogeneous cell populations.

Genome editing provides an alternative approach to lentiviral integrations to perform permanent genetic engineering of cells. Genome editing can be performed using non-nuclease approaches [Barzel, A. et al., Nature 517, 360-364 (2015); Russell, D. W. & Hirata, R. K, Nat Genet 18, 325-330 (1998)], by base editing [Komor, A. C. et al., Nature 533, 420-424 (2016)], or by prime-editing [Anzalone, A. V. et al., Nature (2019)], but the most developed and efficient form of precision engineering in human cells utilizes engineered nuclease-based approaches [Miller, D. G. et al., Mol Cell Biol 23, 3550-3557 (2003); Porteus, M. H. & Baltimore, D., Science 300, 763 (2003); Genovese, P. et al., Nature 510, 235-240 (2014); Urnov, F. D. et al., Nature 435, 646-651 (2005); Porteus, M. H. & Carroll, D., Nature biotechnology 23, 967-973 (2005); Lombardo, A. et al., Nat Methods 8, 861-869 (2011)]. The repurposing of the bacterial CRISPR/Cas9 system for use in human cells [Jinek, M. et al., Science 337, 816-821 (2012); Cong, L. et al., Science 339, 819-823 (2013)] has democratized the field of genome editing because of its ease of use, high activity, and high specificity, especially using high fidelity versions of Cas9 [Vakulskas, C. A. et al., Nat Med 24, 1216-1224 (2018)]. Nuclease-based editing has now entered clinical trials with more on the horizon [Porteus, M. H., N Engl J Med 380, 947-959 (2019)].

Genome editing by combining ribonucleoprotein (RNP, Cas9 protein complexed to synthetic stabilized, single guide RNAs) combined with the use of the non-integrating AAV6 viral vector to deliver the donor template has been shown to be a highly effective system to modify therapeutically relevant primary human cells including HSPCs, T-cells, and induced pluripotent cells [Martin, R. M. et al., Cell Stem Cell 24, 821-828 e825 (2019)]. This approach has shown pre-clinical promise to usher in a new class of medicines for sickle cell disease [Vakulskas, C. A. et al., Nat Med 24, 1216-1224 (2018); Dever, D. P. et al., Nature 539, 384-389 (2016)], SCID-X1 [Pavel-Dinu, M. et al., Nat Commun 10, 1634 (2019); Schiroli, G. et al., Sci Transl Med 9 (2017)] MPS I [Gomez-Ospina, N. et al., Nat Commun 10, 4045 (2019)], chronic granulomatous disease [De Ravin, S. S. et al., Sci Transl Med 9 (2017)], X-linked Hyper IgM [Hubbard, N. et al., Blood 127, 2513-2522 (2016)], and cancer [Eyquem, J. et al., Nature 543, 113-117 (2017)]. The specificity of genome editing, however, means that with current approaches it is not possible to track the output of any specific gene modified cell. The spectrum of non-homologous end joining (NHEJ)-introduced INDELs is also not broad enough to reliably measure clonal dynamics within a population [van Overbeek, M. et al., Mol Cell 63, 633-646 (2016)]. Yet, understanding clonal dynamics within large populations of engineered cells is important and significant in both pre-clinical studies and potentially clinical studies. Therefore, we developed a barcode system for homologous recombination-based genome editing. We applied this system to understand the clonal dynamics of CD34⁺ human HSPCs following transplantation into immunodeficient NSG mice.

We describe TRACE-Seq, a methodology that allows for both correction of disease-specific mutations and for the tracking of contributions of gene targeted HSPCs to single and multi-lineage hematopoietic reconstitution. In brief, we demonstrate: 1) design and production of barcoded AAV6 donor templates using silent in-frame mutations or semi-randomized nucleotides outside the coding region (but inside the homology arms), 2) barcoding the first 9 amino acids of HBB exon 1 with 20,000 possible AAV6 donor templates maintains high gene correction frequencies while preserving robust beta globin expression levels, 3) the ability to track the reconstitution of gene corrected myeloid- and lymphoid-skewed HSPC clones as well as balanced multi-lineage clones, and 4) an analysis pipeline that includes a highly adaptable platform for interpreting and summarizing rich datasets from clonal tracking studies that is deployable as a website accessible to researchers with no coding experience. TRACE-Seq demonstrates that Cas9 RNP and AAV6-mediated gene correction can be used to target a single HSC clone that can then robustly repopulate the myeloid and lymphoid branches of the hematopoietic system. This method and information further supports the translational potential of homologous recombination based approaches for the treatment of genetic diseases of the blood and immune system.

### Methods

### Donor Design and Cloning

### HBB Barcode Donor Libraries:

AAV transfer plasmid with inverted terminal repeats (ITR) from AAV2 that contained 2.4 kb of the HBB gene previously described [Dever, D. P. et al., Nature 539, 384-389 (2016)] was digested with NcoI and BamH1 restriction enzymes (NEB) that resulted in deletion of a 435 bp band and the digested backbone was collected for further subcloning. Double stranded DNA gBlock (IDT) pools with degenerate bases representing silent mutations containing 645 bases of homology were ordered in four separate oligo pools (as detailed below with bold depicting silent mutation region). Four different barcoded dsDNA oligo pools were ordered to maximize potential silent mutations that if all were ordered in the same library would have resulted in amino acid changes to the coding region. Each HBB barcoded dsDNA pool was then digested with NcoI and BamHI resulting in a 435 bp band that was collected and purified. NEB Assembly ligation reactions were performed for 1 hour at 50° C. using digested, gel purified vector. Ligated HBB barcoded donor pools were transformed using NEB DH10B electrocompetent bacteria (NEB C₃₀₂₀K) or XL10-Gold competent cells (Agilent 200315) according to the manufacturer's protocol. At least two times the theoretical maximum number of possible barcoded donor templates were plated to ensure generation of as much diversity as possible. Endotoxin-free maxipreps were generated for AAV6 production and purification. As noted, HBB barcode pool 3 was not included in genome editing experiments because enrichment of the original undigested donor plasmid was seen during sequencing of the plasmid library.

HBB barcode pool 1 (8192 possible unique donor
templates):
(SEQ ID NO: 1)
agaagagccaaggacaggtacggctgtcatcacttagacctcaccctgtgg
agccacaccctagggttggccaatctactcccaggagcagggagggcagga
gccagggctgggcataaaagtcagggcagagccatctattgcttacatttg
cttctgacacaactgtgttcactagcaacctcaaacagacaccatggTNCA
YTTRACNCCNGARGARAARTCNGCAGTCACTgccctgtggggcaaggtgaa
cgtggatgaagttggtggtgaggccctgggcaggttggtatcaaggttaca
agacaggtttaaggagaccaatagaaactgggcatgtggagacagagaaga
ctcttgggtttctgataggcactgactctctctgcctattggtctattttc
ccacccttaggctgctggtggtctacccttggacccagaggttctttgagt
cctttggggatctgtccactcctgatgctgttatgggcaaccctaaggtga
aggctcatggcaagaaagtgctcggtgcctttagtgatggcctggctcacc
tggacaacctcaagggcacctttgccacactgagtgagctgcactgtgaca
agctgcacgtggatcctgagaacttcagggtga
HBB barcode pool 2 (4096 possible unique donor
templates):
(SEQ ID NO: 2)
agaagagccaaggacaggtacggctgtcatcacttagacctcaccctgtgg
agccacaccctagggttggccaatctactcccaggagcagggagggcagga
gccagggctgggcataaaagtcagggcagagccatctattgcttacatttg
cttctgacacaactgtgttcactagcaacctcaaacagacaccatggTNCA
YTTRACNCCNGARGARAARAGYGCAGTCACTgccctgtggggcaaggtgaa
cgtggatgaagttggtggtgaggccctgggcaggttggtatcaaggttaca
agacaggtttaaggagaccaatagaaactgggcatgtggagacagagaaga
ctcttgggtttctgataggcactgactctctctgcctattggtctattttc
ccacccttaggctgctggtggtctacccttggacccagaggttctttgagt
cctttggggatctgtccactcctgatgctgttatgggcaaccctaaggtga
aggctcatggcaagaaagtgctcggtgcctttagtgatggcctggctcacc
tggacaacctcaagggcacctttgccacactgagtgagctgcactgtgaca
agctgcacgtggatcctgagaacttcagggtga
HBB barcode pool 3 (16384 possible unique donor
templates):
(SEQ ID NO: 3)
agaagagccaaggacaggtacggctgtcatcacttagacctcaccctgtgg
agccacaccctagggttggccaatctactcccaggagcagggagggcagga
gccagggctgggcataaaagtcagggcagagccatctattgcttacatttg
cttctgacacaactgtgttcactagcaacctcaaacagacaccatggTNCA
YCTNACNCCNGARGARAARTCNGCAGTCACTgccctgtggggcaaggtgaa
cgtggatgaagttggtggtgaggccctgggcaggttggtatcaaggttaca
agacaggtttaaggagaccaatagaaactgggcatgtggagacagagaaga
ctcttgggtttctgataggcactgactctctctgcctattggtctattttc
ccacccttaggctgctggtggtctacccttggacccagaggttctttgagt
cctttggggatctgtccactcctgatgctgttatgggcaaccctaaggtga
aggctcatggcaagaaagtgctcggtgcctttagtgatggcctggctcacc
tggacaacctcaagggcacctttgccacactgagtgagctgcactgtgaca
agctgcacgtggatcctgagaacttcagggtga
HBB barcode 4 (8192 possible unique donor
templates):
(SEQ ID NO: 4)
agaagagccaaggacaggtacggctgtcatcacttagacctcaccctgtgg
agccacaccctagggttggccaatctactcccaggagcagggagggcagga
gccagggctgggcataaaagtcagggcagagccatctattgcttacatttg
cttctgacacaactgtgttcactagcaacctcaaacagacaccatggTNCA
YCTNACNCCNGARGARAARAGYGCAGTCACTgccctgtggggcaaggtgaa
cgtggatgaagttggtggtgaggccctgggcaggttggtatcaaggttaca
agacaggtttaaggagaccaatagaaactgggcatgtggagacagagaaga
ctcttgggtttctgataggcactgactctctctgcctattggtctattttc
ccacccttaggctgctggtggtctacccttggacccagaggttctttgagt
cctttggggatctgtccactcctgatgctgttatgggcaaccctaaggtga
aggctcatggcaagaaagtgctcggtgcctttagtgatggcctggctcacc
tggacaacctcaagggcacctttgccacactgagtgagctgcactgtgaca
agctgcacgtggatcctgagaacttcagggtga

AAVS1 barcode donor libraries:

AAVS1 barcode libraries were generated similarly to HBB libraries. Briefly, degenerate nucleotides (following the pattern “VHDBVHDBVHDB (SEQ ID NO: 5),” in order to minimize homopolymer stretches as described [Davidsson, M. et al., Sci Rep 6, 37563 (2016)] were introduced by PCR 3′ of the mTagBFP2 reporter cassette. pAAV-MCS plasmid (Agilent Technologies) containing ITRs from AAV serotype 2 (AAV2) was digested with Notl and barcode-containing PCR fragments were assembled into the backbone using NEB Assembly using the following primers, prior to transformation with XL10-Gold competent cells (Agilent 200315):

Insert_Fw1:
(SEQ ID NO: 6)
CCATCACTAGGGGTTCCTGCGGCCGCCACCGTTTTTCT
Insert_Rv1:
(SEQ ID NO: 7)
TTAATTAAGCTTGTGCCCCAGTTTGCTAGG
Insert_Fw2:
(SEQ ID NO: 8)
TGGGGCACAAGCTTAATTAAVHDBVHDBVHDBCTCGAGGGCGC
Insert_Rv2:
(SEQ ID NO: 9)
CCATCACTAGGGGTTCCTGCGGCCGCAGAACTCAGGAC

### AAV6 Production and Purification

HBB barcoded recombinant adeno-associated virus serotype (AAV6) six vectors were produced and purified as previously described [Bak, R. O. et al., Nat Protoc 13, 358-376 (2018)]. Briefly, 293FT cells (Life Technologies) were seeded at 15 million cells per dish in a total of ten 15-cm dishes one to two days before transfection (or until they are 80-90% confluent). One 15-cm dish was transfected with 6 μg ITR-containing HBB barcoded donor plasmid pools 1˜4 and 22 μg pDGM6. Cells were incubated for 48-72h until collection of AAV6 from cells by three freezes-thaw cycles. AAV6 vectors were purified on an iodixanol density gradient, AAV6 vectors were extracted at the 60-40% iodixanol interface, and dialyzed in PBS with 5% sorbitol with 10K MWCO Slide-A-Lyzer G2 Dialysis Cassette (Thermo Fisher Scientific). Finally, vectors were added to pluronic acid to a final concentration of 0.001%, aliquoted, and stored at −80° C. until use. AAV6 vectors were tittered using digital droplet PCR to measure the number of vector genomes as described previously [Aurnhammer, C. et al., Hum Gene Ther Methods 23, 18-28 (2012)]. AAVS1 barcoded AAV6 donors were produced as described above but purified using a commercial purification kit (Takara Bio #6666).

### CD34⁺ Hematopoietic Stem and Progenitor Cell Culture

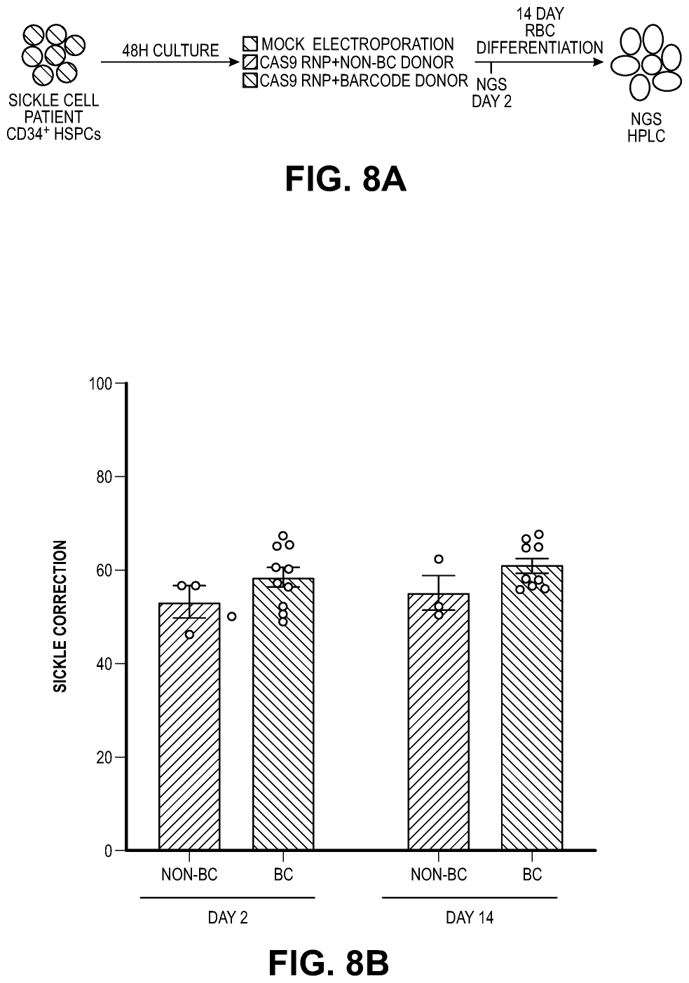

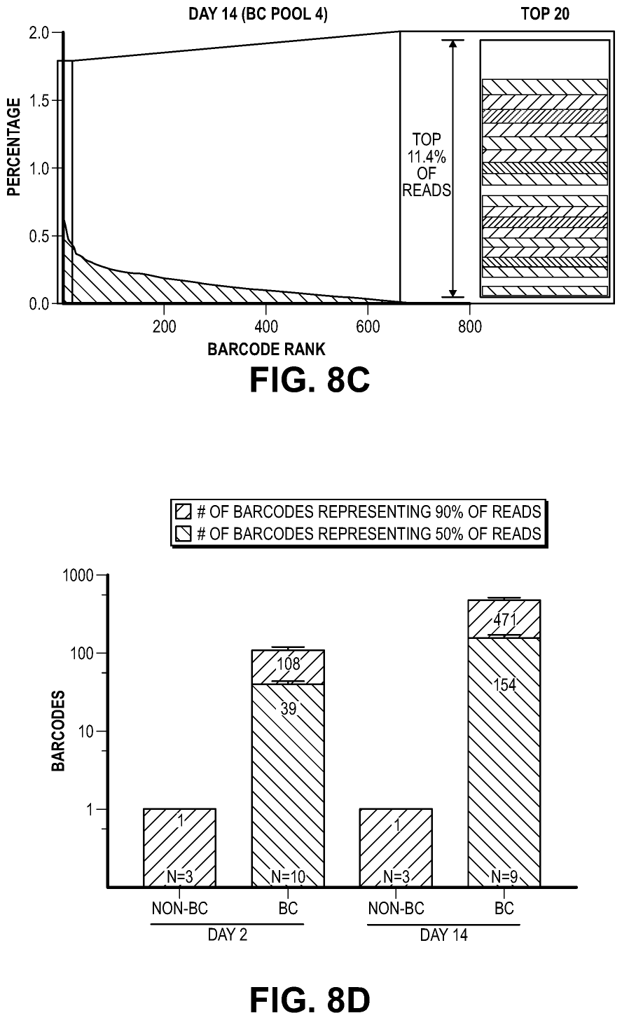

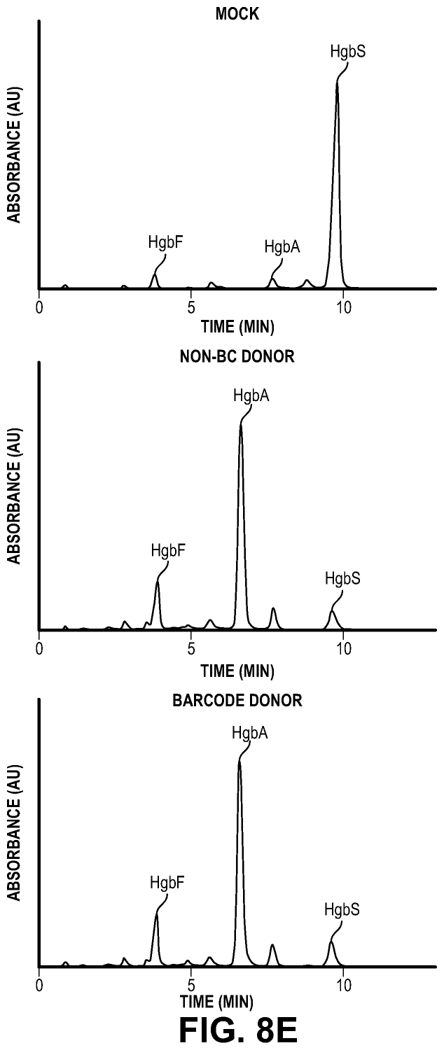

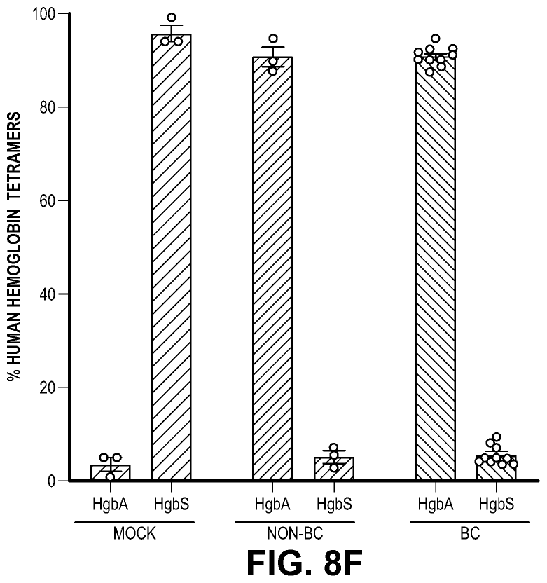

All CD34⁺ cells used in these experiments were cultured as previously described [Bak, R. O. et al., Nat Protoc 13, 358-376 (2018)]. In brief, cells were cultured in low-density conditions (<250,000 cells/mL), low oxygen conditions (5% O₂), in SFEMII (Stemcell Technologies) or SCGM (CellGenix) base media supplemented with 100 ng/mL of TPO, SCF, FLT3L, IL-6 and the small molecule UM-171 (35 nM). For in vitro studies presented in FIG. 8, CD34⁺ cells from sickle cell disease patients were obtained as a kind gift from Dr. John Tisdale at the National Institute of Health (that were mobilized with plerixafor in accordance with their informed consent) or from routine non-mobilized peripheral blood transfusions at Stanford University under informed consent. For in vivo studies presented, cord blood-derived CD34⁺ cells were purchased from AllCells or Stemcell Technologies and were thawed according to the manufacturer's recommendations.

### Cas9/sgRNA and AAV6-Mediated Genome Editing

All experiments in these studies used the R691A HiFi Cas9 mutant [Vakulskas, C. A. et al., Nat Med 24, 1216-1224 (2018)] (IDT and Aldevron), and chemically synthesized guide RNA (sgRNA) [Hendel, A. et al., Nature biotechnology 33, 985-989 (2015)] (Synthego). The guide sequences were as follows: HBB: 5′-CTTGCCCCACAGGGCAGTAA-3′ (SEQ ID NO: 10) and AAVS1: 5′-GGGGCCACTAGGGACAGGAT-3′ (SEQ ID NO: 11). Genome editing experiments using Cas9/sgRNA and AAV6 were performed as previously described [Bak, R. O. et al., Nat Protoc 13, 358-376 (2018)]. In brief, CD34⁺ HSPCs were thawed and plated for 48h to allow for recovery of freezing process and pre-stimulation of cell cycle. CD34⁺ HSPCs were then electroporated in 100 μl electroporation reaction buffer P3 (Lonza) with 30 μg HiFi Cas9 and 16 μg MS sgRNA (pre-complexed for 10 minutes at room temperature; HiFi RNP). HSPCs were resuspended with HiFi RNP in P3 buffer and electroporated using program DZ-100 on the Lonza 4D nucleofector. Immediately following electroporation, CD34⁺ HSPCs were transduced with HBB-specific AAV6 barcoded donor template libraries at 2500-5000 vector genomes per cell and 20000 vector genomes per cell for AAVS1-specific AAV6 barcoded libraries. 12-16h post transduction, targeted cells were washed and resuspended in fresh media and allowed to culture for additional 24-36h, with a total manufacturing time less than 96h.

### In Vitro Erythrocyte Differentiation of HBB-Targeted CD34⁺ HSPCs

SCD-HSPCs were targeted with either the therapeutic AAV6 donor (with one sequence) or the HBB barcoded AAV6 donor template library and subjected to the in vitro erythrocyte differentiation protocol two days post targeting as previously described [Vakulskas, C. A. et al., Nat Med 24, 1216-1224 (2018); Dulmovits, B. M. et al., Blood 127, 1481-1492 (2016); Hu, J. et al., Blood 121, 3246-3253 (2013)]. Base medium was supplemented with 100U/mL of penicillin-streptomycin, 10 ng/mL SCF, 1 ng/mL IL-3 (PeproTech), 3U/mL erythropoietin (eBiosciences), 200 μg/mL transferrin (Sigma-Aldrich), 3% antibody serum (heat-inactivated from Atlanta Biologicals, Flowery Branch, Ga., USA), 2% human plasma (umbilical cord blood), 10 μg/mL insulin (Sigma Aldrich) and 3U/mL heparin (Sigma-Aldrich). Briefly, targeted HSPCs were differentiated into erythrocytes using a three-phase differentiation protocol that lasted 14-16 days in culture. The first phase of erythroid differentiation corresponded to days 0-7 (day 0 being day 2 after electroporation). During the second phase of differentiation, corresponding to days 7-10, IL-3 was discontinued from culture medium. In the third and final phase, corresponding to days 10-16, transferrin was increased to 1 mg/mL. Differentiated cells were then harvested for analysis of hemoglobin tetramers by cation-exchange high performance liquid chromatography.

### Hemoglobin Tetramer Analysis Via Cation-Exchange HPLC

Hemoglobin tetramer analysis was performed as previously described [Vakulskas, C. A. et al., Nat Med 24, 1216-1224 (2018)]. Briefly, red blood cell pellets were flash frozen post differentiation until tetramer analysis where pellets were then thawed, lysed with 3 times volume of water, incubated for 15 minutes and then sonicated for 30 seconds to finalize the lysing procedure. Cells were then centrifuged for 5 minutes at 13,000 rpm and used for input to analyze steady-state hemoglobin tetramer levels. Transfused blood from sickle cell disease patients was always used to ascertain the retention time of sickle, adult and fetal human hemoglobin.

Transplantation of Targeted CD34⁺ HSPCs into NSG Mice

Six to eight week old immunodeficient NSG female mice were sublethally irradiated with 200cGy 12-24h before injection of cells. For primary transplants, 2-4×10⁵ targeted CD34⁺ HSPCs were harvested two days post electroporation, spun down at 300 g, and resuspended in 25 μl PBS before intrafemoral transplantation into the right femur of female NSG mice. For secondary transplants, mononuclear cells (MNCs) were harvested from primary transplanted NSG mice, and half of the total MNCs were used to transplant one sublethally irradiated female NSG mouse via tail vein injection.

### Analysis of Human Engraftment and Fluorescent Activated Cell Sorting

16-18 weeks following transplantation of targeted HSPCs, mice were euthanized, bones (2× femurs, 2× pelvis, 2× tibia, sternum, spine) were collected and crushed as previously described [Dever, D. P. et al., Nature 539, 384-389 (2016); Bak, R. O. et al., Nat Protoc 13, 358-376 (2018)]. MNCs were harvested by ficoll gradient centrifugation and human hematopoietic cells were identified by flow cytometry using the following antibody cocktail: HLA-A/B/C FITC (clone W6/32, Biolegend), mouse CD45.1 PE-CY7 (clone A20, Thermo Scientific), CD34 APC (clone 581, Biolegend), CD33 V450 (clone WM53, BD Biosciences), CD19 Percp5.5 (clone HIB19, BD Biosciences), CD10 APC-Cy7 (HI10a, Biolegend), mTer119 PeCy5 (clone Ter-119, Thermo Scientific), and CD235a PE (HIR2, Thermo Scientific). For mice transplanted with AAVS1-edited HSPCs, the following cocktail was used: HLA-A/B/C FITC (clone W6/32, Biolegend), mouse CD45.1 PE-CY7 (clone A20, Thermo Scientific), CD34 APC (clone 581, Biolegend), CD33 PE (clone WM53, BD Biosciences), CD19 BB700 (clone HIB19, BD Biosciences), CD3 APC-Cy7 (clone SK7, BD Biosciences). For AAVS1-edited HSPCs, CD33^{Hi} and CD33^{Mid} were sorted individually, however the data were aggregated for analysis.

Human hematopoietic cells were identified as HLA-A/B/C positive and mCD45.1 negative. The following gating scheme was used to sort cell lineages to be analyzed for barcoded recombination alleles: Myeloid cells (CD33⁺), B Cells (CD19⁺), HSPCs (CD10⁻, CD34⁺, CD19⁻, CD33⁻), and erythrocytes (Ter119⁻, mCD45.1⁻, CD19⁻, CD33-, CD10⁻, CD235a⁺). Sorted cells were spun down, genomic DNA was harvested using QuickExtract (Lucigen), and was saved until library preparation and sequencing.

### Sequencing Library Preparation

Harvested cells were lysed using QuickExtract DNA Extraction Solution (Lucigen, Cat. No. QE09050) following manufacturers protocol. Based on the starting cell count, 0.5-1 μL QuickExtract lysate was used for PCR. All PCRs for library preparation were carried out using Q5 High-Fidelity 2× Master Mix (NEB, Cat. No. M0492L). An initial enrichment amplification of 15 cycles was followed with a second round of PCR using unique P5 and P7 indexing primer combinations for 15 cycles and purified using 1.8×SPRI beads. For nested PCR, an initial amplification of 30 cycles was used. PCR products were analyzed by gel electrophoresis and purified using 1×SPRI beads.

PCR products were normalized, pooled and then gel extracted using the QIAEX II Gel Extraction Kit (Qiagen, Cat. No. 20051). The resulting libraries were sequenced using both Illumina Miseq (2×150 bp paired end) and Illumina HiSeq 4000 (2×150 bp paired end) platforms. Illumina HiSeq 4000 sequencing were performed by Novogene Corporation.

### Index Switching Correction of False Positive NGS Reads

We utilized two independent methods to determine the incidence of index-switching present in samples that were run on a HiSeq 4000 [Costello, M. et al., BMC Genomics 19, 332 (2018); Sinha, R. et al., bioRxiv, 125724 (2017)]. In one approach, we calculated the number of contaminating reads between two different amplicons sequenced in the same pool. As a second approach, we utilized the algorithm developed by Larrson et al. to estimate the fraction of reads which were spread to other samples through index switching [Larsson, A. J. M. et al., Nat Methods 15, 305-307 (2018)]. Both of these methods yielded an index switching incidence of 0.3%. We performed a conservative correction for this by subtracting 0.3%×[#Barcode Reads] from each barcode in each sample. We performed this correction after clustering as described in Extended Data FIG. 1.

### Statistical Analysis

All statistical tests used in this study were performed using GraphPad Prism 7/8 or R version 3.6.1. For comparing the average of two means, we used the Student's t-test to reject the null hypothesis (P<0.05).

### Results

### Design, Production, and Validation of Barcoded AAV6 Donor Templates for Targeting the HBB Gene in Human HSPCs

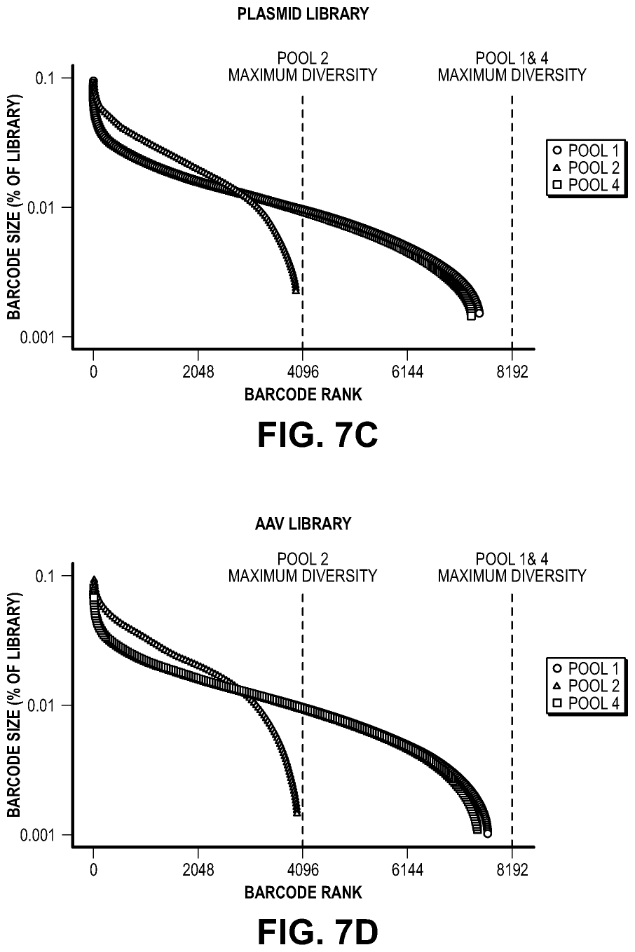

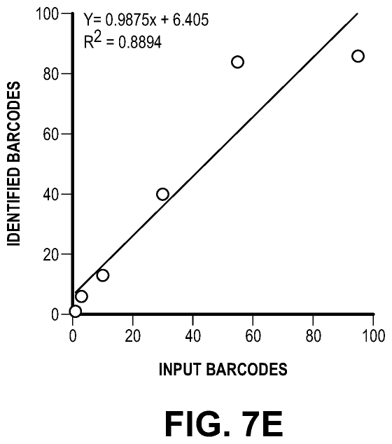

We previously developed an HBB AAV6 homologous donor template that corrects the sickle cell disease-causing mutation in HSPCs with high efficiencies [Dever, D. P. et al., Nature 539, 384-389 (2016)]. Using this AAV6 donor as a template, we designed an HBB barcoded AAV6 donor library with the ability to: 1) correct the E6V sickle mutation, 2) preserve the reading frame of the beta globin gene, and 3) generate enough sequence diversity to track cellular events on the clonal level (throughout the manuscript we will consider unique barcodes representative of cellular clones, with the caveat that clone counts may be overestimated due to bi-allelic targeting of two barcodes into the genome of a single cell). We designed the donor pool to contain mixed nucleotides that encode silent mutations within the first 9 amino acids of the HBB coding sequence (“VHLTPEEKS” (SEQ ID NO: 12), FIG. 7a). Using this strategy, we designed double stranded DNA oligos that contained the library of nucleotide sequences and cloned four separate pools of donors with a theoretical maximum number of 36,864 in-frame, synonymous mutations (FIG. 7b).

To ensure that the initial plasmid library reached the theoretical maximum diversity with near-equal representation of all sequences, we performed amplicon sequencing on the initial plasmid pools. Sequencing of HBB barcoded pools 1, 2, and 4 (FIG. 7a, bottom) revealed a wide distribution of sequences with no evidence of any highly overrepresented barcodes (FIG. 7c). Barcode pool 3 was eliminated for further study, because it was contaminated with uncut vector control and therefore skewed barcode diversity. After validating that the plasmid pools were diverse and lacked enrichment of any one sequence, we used the HBB barcoded library plasmid pools 1, 2, and 4 to produce libraries of AAV6 homologous donor templates. After generating barcoded AAV6 donor libraries, we performed amplicon-based NGS to determine the diversity and distribution of sequences. Similar patterns were observed, suggesting standard AAV6 production protocols do not introduce donor template bias in the barcoded pool (FIG. 7d).

### Establishing Thresholds for HBB Barcode Quantification

Understanding the clonal dynamics of hematopoietic reconstitution through sequencing requires the ability to differentiate between low frequency barcodes and noise introduced by sequencing error. Therefore, we used a modified version of the TUBAseq pipeline to cluster cellular barcodes and differentiate between sequencing error and bona-fide barcode sequences [Rogers, Z. N. et al., Nat Methods 14, 737-742 (2017)]. Briefly, we merged paired-end fastq files using the PEAR algorithm with standard parameters [Zhang, J. et al., Bioinformatics 30, 614-620 (2014)], and then aligned reads to the human HBB gene. Reads were binned into three categories: unmodified alleles (wildtype), non-homologous end joining (NHEJ) alleles, and homologous recombination (HR) alleles. Reads were classified as unmodified if they aligned to the reference HBB gene with no genome edits. Reads were classified as NHEJ if there were any insertions or deletions within 20 bp of the cut site, and if anchor bases (PAM-associated bases changed after successful HR) were unmodified (FIG. 7a). Finally, reads were classified as HR if they had modified anchor bases and were not classified as NHEJ (FIG. 7a). All subsequent analyses were performed exclusively on the HR reads.

To differentiate between bona fide barcodes and sequencing errors, variable barcode regions and non-variable training regions were extracted from the HR reads and TUBAseq was used to train an error model and cluster similar barcodes together using the DADA2 algorithm [Rogers, Z. N. et al., Nat Methods 14, 737-742 (2017)]. We chose a DADA2 clustering omega parameter of 10⁻⁴⁰ because: 1) we found that at this omega value, the number of unfiltered barcodes called began to reach the minimum number of barcodes called per sample as omega was decreased, and 2) we found that varying this parameter did not ultimately affect the number or sequence of called barcodes after filtering (described subsequently) for samples with known barcode content (data not shown).

In order to benchmark our analysis pipeline, we cultured individual barcoded bacterial plasmid colonies in 96 well plates and generated pooled plasmid libraries to generate a set of ground-truth samples with known barcode content. These libraries were spiked into untreated human gDNA and were subjected to our optimized amplicon sequencing and analysis pipeline. We found that clustering eliminated more than 97% of low-level noise barcodes across all samples with known barcode content, but left a small percentage of low-level barcodes in the clustered barcode set (data not shown). Using the ground-truth samples, we determined a “high confidence” barcode threshold of 0.5%, which allowed us to quantitatively recover the expected numbers of barcodes (R²=0.89) (FIG. 7e).

Overall, our pipeline allowed us to process raw amplicon sequencing data and generate a set of barcodes unlikely to contain spurious signals. Conceptually, we extracted barcodes from each read and eliminated barcodes which appeared to be derived from sequencing or other error using a clustering-based methodology and evidence-based filtering heuristics, resulting in a set of high-confidence barcodes with which we performed further analyses.

Barcoding HBB Exon 1 with In-Frame Silent Mutations Preserves Hemoglobin Expression while Allowing Cell Tracking within a Heterogeneous Population

To evaluate whether the barcoded AAV6 donor libraries preserved the open reading frame of HBB following targeted integration, we compared HSPCs targeted with a non-barcoded homologous donor (containing a single corrective AAV6 genome [Dever, D. P. et al., Nature 539, 384-389 (2016)]; non-BC) or a barcode donor library (BC) as illustrated in FIG. 8a. We performed gene-targeting experiments by electroporating HiFi Cas9 and HBB-specific chemically modified guide sgRNAs [Hendel, A. et al., Nature biotechnology 33, 985-989 (2015)] into primary CD34⁺ HSPCs isolated from patients with sickle cell disease (which contained the E6V point mutation). We observed similar gene correction efficiencies between HSPCs targeted with non-BC and BC donors as quantified by amplicon-based next generation sequencing from approximately 1000 cells from each timepoint (FIG. 8b). To assess barcode diversity, we ranked barcodes by read percentages from largest to smallest for each treatment group (FIG. 8c). Focusing specifically on the top 20 barcodes in the representative example in FIG. 8c, it is evident that even with a relatively small sample, we observe a fairly even distribution of barcodes, with no evidence of extreme overrepresentation from any particular sequences. We calculated the number of the most abundant barcodes comprising 50% and 90% of total HR reads as a measure of sequence diversity. As expected, the single non-BC donor sample contained one barcode (the corrected E6V sequence) along with intentional synonymous mutations [Dever, D. P. et al., Nature 539, 384-389 (2016)] that represented >94% of reads (FIG. 8d). Of note, the remaining reads appeared to be sequencing/PCR artifacts as they often contained nonsynonymous mutations in the HBB reading frame (data not shown). In contrast, the 90^{th} percentile of barcode reads in BC donor targeted cells contained a mean of 107.7±9.6 barcodes at day 2 and 471.0±54.1 at day 14 (FIG. 8d). These unique barcode counts were not surprising given the limited numbers of input cells analyzed, and the additional complexity of performing nested PCR reactions to avoid contamination from unintegrated (episomal) AAV6 donor genomes, especially at early timepoints before the cells could undergo many rounds of division. Indeed, by aggregating together all experimental replicates treated with BC donors, the 90^{th} percentile of barcode reads contained >3200 barcodes (data not shown), suggesting barcode identification was limited by sampling depth. Importantly, the barcodes observed in the BC donor treated samples preserved the HBB coding sequence even though their sequences varied greatly (data not shown). These results are consistent with the notion that targeting HSPCs with a BC donor produces a diverse pool of HSPCs capable of correcting the E6V sickle mutation, and that diversity is maintained within a two-week period of in vitro culture.

While the sequencing data suggest that the HSPCs targeted with the BC donors exhibit robust E6V gene correction frequencies, the introduction of silent mutations may interfere with hemoglobin protein expression. To assess this possibility, we performed in vitro erythroid differentiation of non-BC and BC targeted HSPCs and collected red blood cell pellets for HPLC analysis of hemoglobin tetramer formation. While the unedited mock sample contained >90% sickle hemoglobin (HgbS) (of total hemoglobin), HSPCs targeted with non-BC or BC AAV6 donors both exhibited>90% adult hemoglobin (HgbA) protein production (FIG. 8e-f). These results suggest the silent mutations introduced by the BC donor had no significant negative influence on overall translation efficiency, despite being produced from a diverse pool of >450 unique sequences in the bulk-edited population.

### TRACE-Seq Reveals Long-Term Engraftment of Lineage-Specific and Bi-Lineage Potent HBB Targeted Hematopoietic Stem and Progenitor Cells

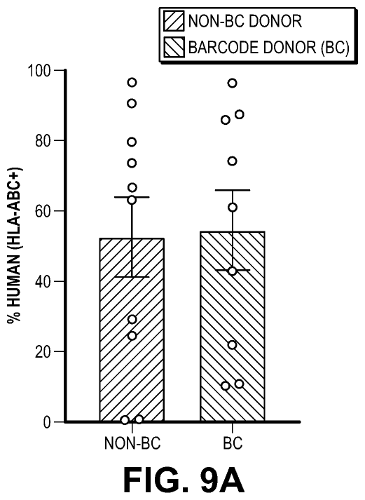

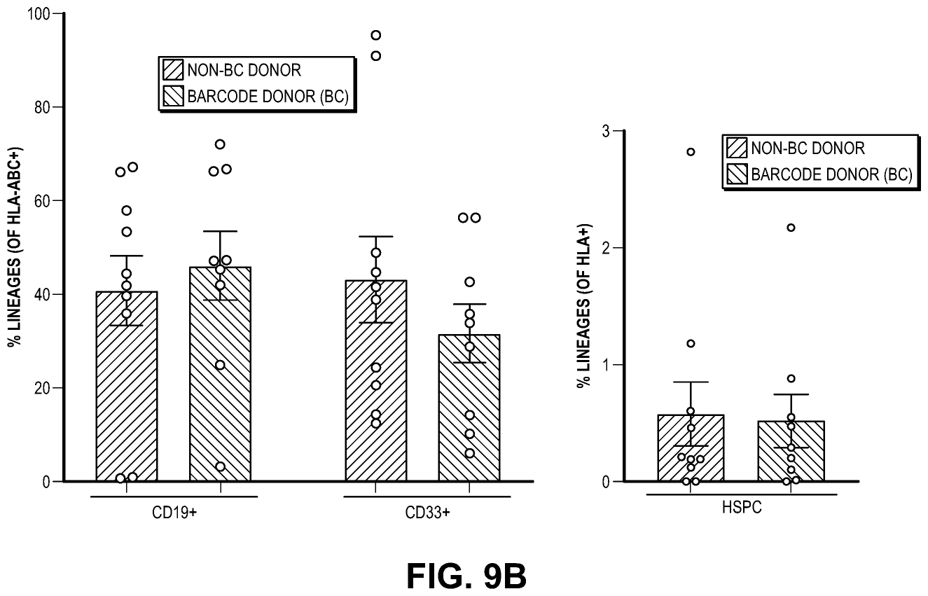

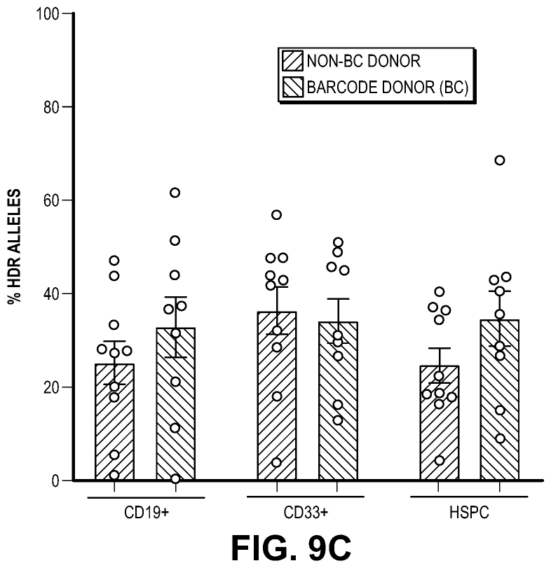

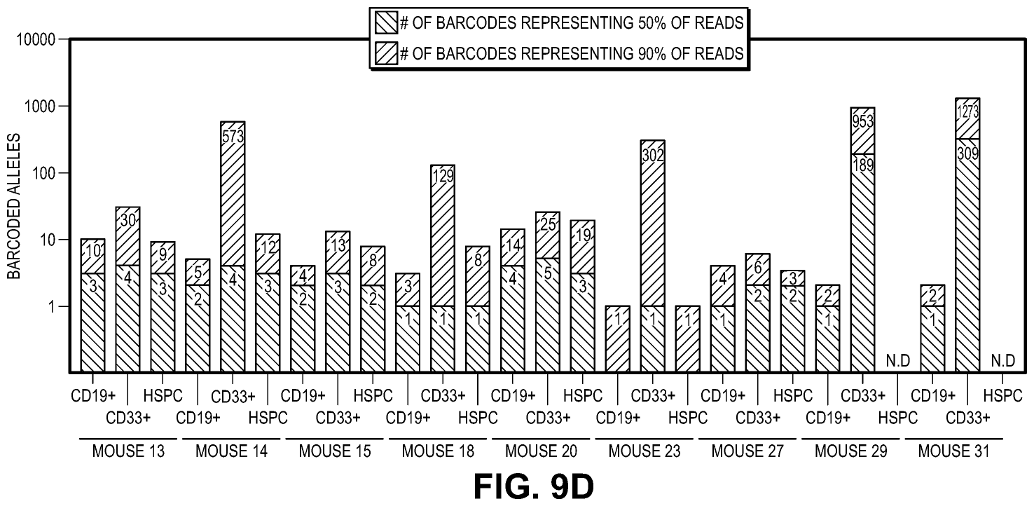

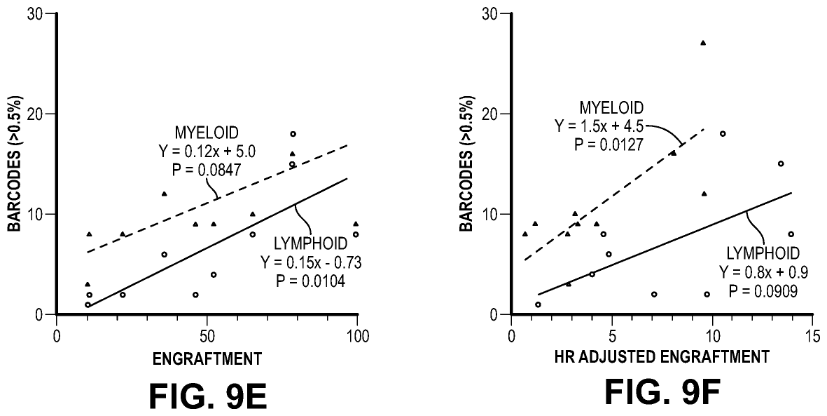

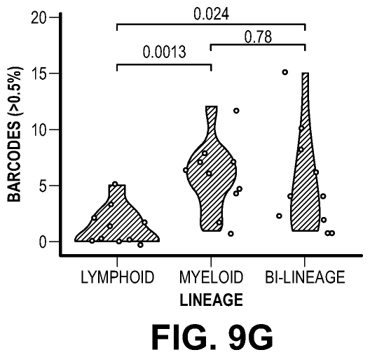

In addition to correcting the E6V mutation and restoring HgbA expression, barcoded AAV6 donors can be utilized to label and track cells in a heterogeneous pool of HSPCs. To track cellular lineages in a pool of HBB-labeled HSPCs, we transplanted BC and non-BC control targeted cord blood CD34⁺ HSPCs via intra-femoral injection into sublethally irradiated adult female NSG recipient mice (2-4×10⁵ cells per mouse from n=6 total cord blood donors). Upon sacrifice (16-18 weeks post-engraftment), mice in both transplantation groups exhibited no statistically significant differences in total human engraftment (46±10.4 vs. 50±10.1, non-BC and BC, respectively, FIG. 9a). Similarly, no significant differences were seen between non-BC and BC mice in terms of lineage reconstitution of the human cells engrafted, which mainly consisted of B cells (CD19⁺), myeloid cells (CD33⁺) or HSPCs (CD19⁻CD33⁻CD10⁻CD34⁺) (FIG. 9b).

To evaluate the efficiency of non-BC or BC gene targeting in long-term engrafting HSPCs, bone marrow MNCs were sorted by flow cytometry into lineages CD19⁺ and CD33⁺, as well as the multipotent HSPC (CD19⁻CD33⁻CD10⁻CD34⁺) populations (data not shown). We performed amplicon based NGS to quantify the proportions of gene targeted alleles relative to total editing events that included NHEJ and unmodified alleles. We did not detect any significant differences in the efficiency of HDR within any of these subsets between non-BC and BC donors (FIG. 9c).

Because there was robust engraftment of HBB targeted alleles in the BC mice, we were able to track the recombination alleles within the lymphoid, myeloid, and multipotent HSPC subpopulations. We analyzed cells from a total of 9 mice sorted on lymphoid (CD19⁺), myeloid (CD33⁺), and HSPC (CD19⁻CD33⁻CD10⁻CD34⁺) markers. 130.6±62.3 unique barcodes accounted for 90% of the reads with a median of 2 unique barcodes accounting for 50% or the sequencing reads from each group (FIG. 9d). Barcodes in all three sorted populations exhibited less diversity than was observed in vitro, indicating that there was a reduction in clonal complexity following engraftment into mice (data not shown). For example, the CD19⁺ compartment from Mouse 18 contained over 60 total clones passing our thresholds, with a majority of reads coming from a single barcode (data not shown). The number of high confidence barcodes (>0.5% of reads) was correlated with total human engraftment in the lymphoid compartment and a similar trend was observed in the myeloid compartment (p=0.08) (FIG. 9e). The same trend was observed when we correlated barcodes with lineage specific engraftment adjusted for HR frequency (FIG. 9f). When we subdivided these more abundant barcodes into alleles that contributed to lymphoid only, myeloid only, or bi-lineage output within the mice, we observed fewer barcodes generated from lymphoid-skewed compared to myeloid-skewed or bi-lineage HSPCs (p=0.0013 and p=0.024, respectively, FIG. 9g). These data suggest that Cas9/sgRNA and AAV6-mediated HBB gene targeting occurs in multipotent HSPCs as well as lineage-restricted HSPCs.

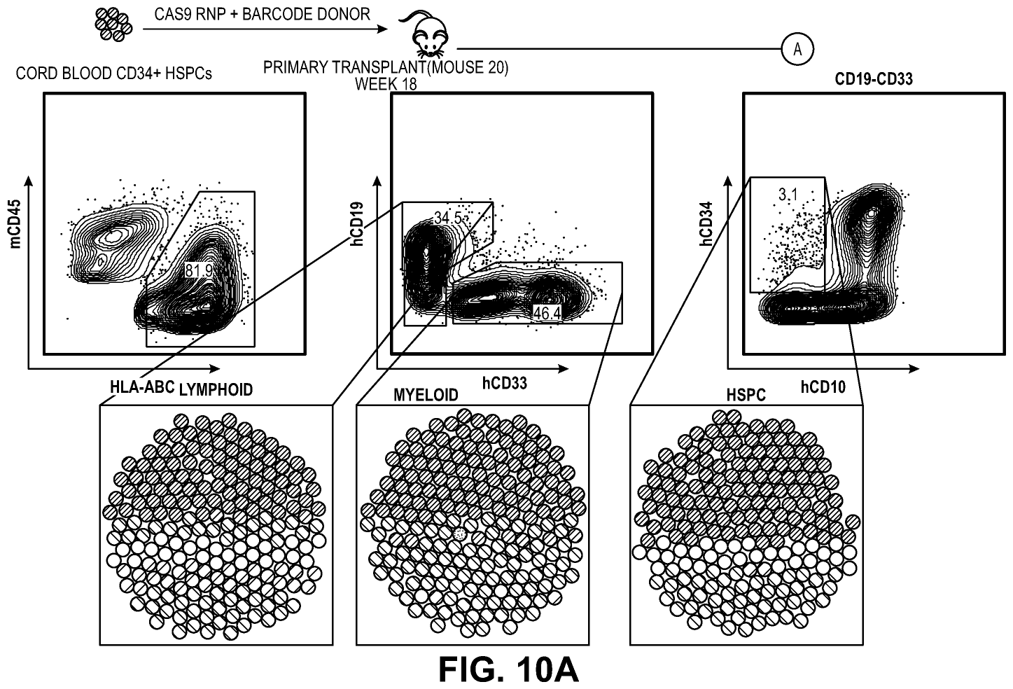

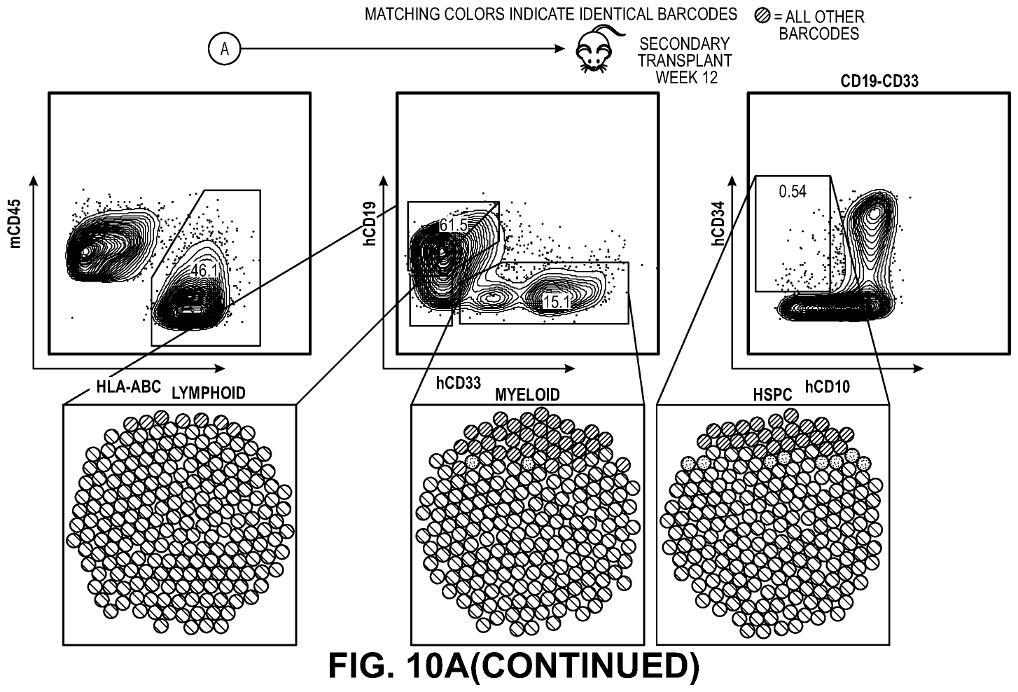

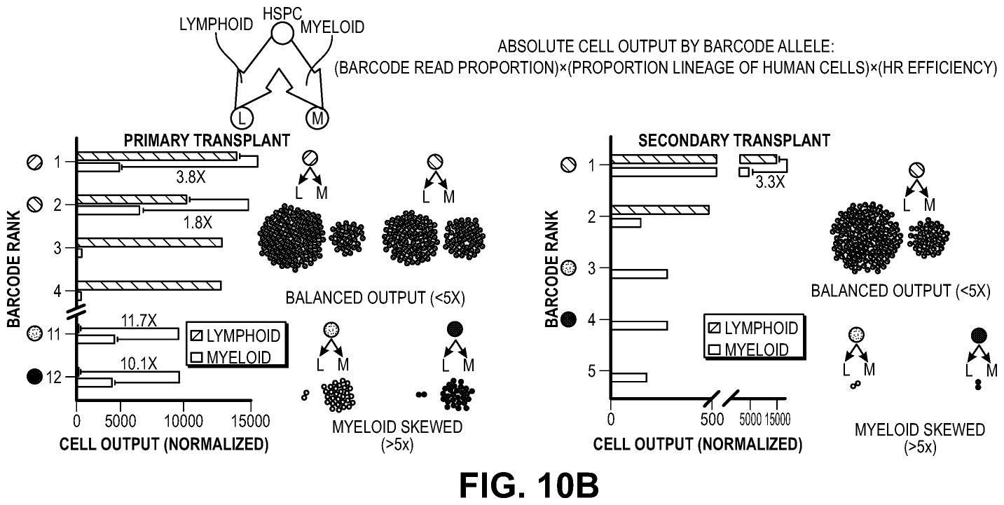

The gold standard for defining human long-term hematopoietic stem cell (LT-HSC) activity is to perform secondary transplants into another sublethally irradiated NSG mouse [Doulatov, S. et al., Cell Stem Cell 10, 120-136 (2012)]. Therefore, we compared the TRACE-Seq dynamics of a primary recipient versus a secondary recipient in mouse 20 that exhibited very high engraftment (>80% human cell engraftment). While mouse 20 had a total of 17 lymphoid and 56 myeloid clones contributing to the engraftment of gene targeted HBB cells, the majority of differentiated cell output was from relatively few clones (FIG. 10a, left panel). Four lymphoid and five myeloid lineage barcodes accounted for 50% of the reads from each population. This trend was consistent between all mice analyzed (data not shown) with each mouse displaying a unique set of HBB barcodes that all maintained the coding region (data not shown). Barcode reads from the same sorted cell populations from the secondary mouse transplant revealed further reductions in clonal diversity, almost to a monoclonal state, with a single clone representing 80% or more of reads in both lymphoid and myeloid lineages (FIG. 10a, right panel, dark blue). Interestingly, the dominant clone in the secondary transplant was not the most abundant clone in the primary mouse as it only represented 10.9% of lymphoid and 16% of myeloid alleles.

To understand the contribution of each clone to the absolute number of differentiated hematopoietic cells in the mouse bone marrow, we took into consideration the following parameters: 1) the fraction of unique barcode reads assigned to each clone, 2) the relative contribution of the lineage where each clone was detected to the entire graft, and 3) gene targeting frequencies (FIG. 10b). This analysis reveals clones that are lymphoid skewed (brown and red, FIG. 10a), myeloid skewed (purple and light green), as well as clones exhibiting balanced hematopoiesis (dark blue). We defined skewing as having a >5-fold difference in proportion between lymphoid and myeloid cells. Perhaps the most interesting observation from this analysis was that the more balanced hematopoietic clone (dark blue) was responsible for a great majority of secondary engraftment/repopulation (FIG. 10b, right). Interestingly, while this clone contributed>80% of the engraftment of HBB targeted cells, there were still observable myeloid lineage-skewed clones present in the secondary transplant. This analysis also revealed barcode sequences that produced highly correlated read frequencies (±2% read proportions) in both primary and secondary transplants, consistent with bi-allelic gene targeting in the same long-term HSPC (FIG. 10b, purple and light green barcodes).

### TRACE-Seq by Barcoding AAV6 Donor Templates Outside the Coding Region Allows for Clonal Tracking of AAVS1 Targeted HSPCs

To test that the barcoding scheme (inside the coding region), library diversity (maximal theoretical diversity of 36,864 HBB barcodes), and/or the gene being targeted (HBB) did not bias our results, we developed a strategy to target AAVS1 with barcoded SFFV-BFP-PolyA AAV6 donor libraries (data not shown). We designed the AAVS1 barcoded variable region within the 3′ untranslated region of the BFP expression cassette so the barcode would be in the genomic DNA as well as mRNA. Using a design that prevents mononucleotide runs that can potentially increase sequencing error [Davidsson, M. et al., Sci Rep 6, 37563 (2016)], a 12 nucleotide variable barcode region resulted in a theoretical maximal barcoded AAV6 pool of 531,441 different homologous donor templates (data not shown). Using such a large pool allowed us to rule out the possibility that the numbers of barcodes observed in the HBB system is artificially limited by the smaller diversity of the HBB barcode pool. As with the HBB pipeline (FIG. 7e), we benchmarked our ability to differentiate sequencing error from legitimate barcodes by choosing parameters and thresholds that resulted in a high correlation between known numbers of input barcodes and barcodes identified through TRACE-seq (data not shown).

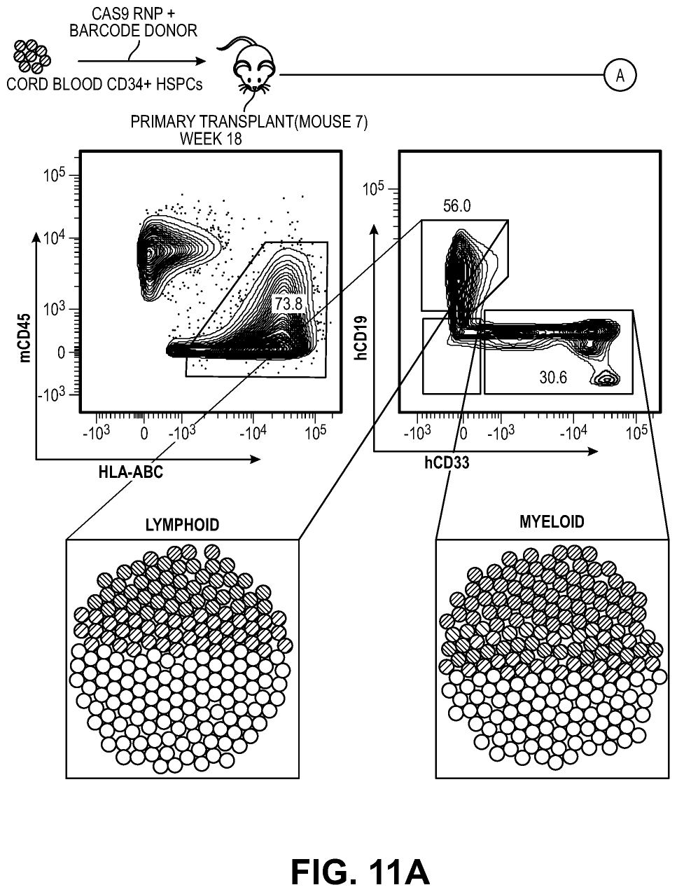

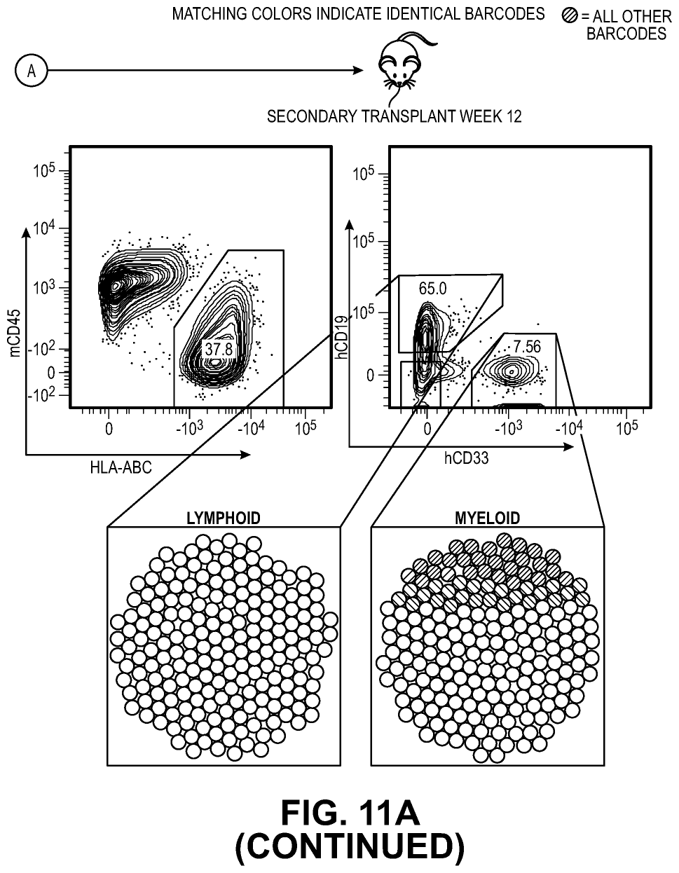

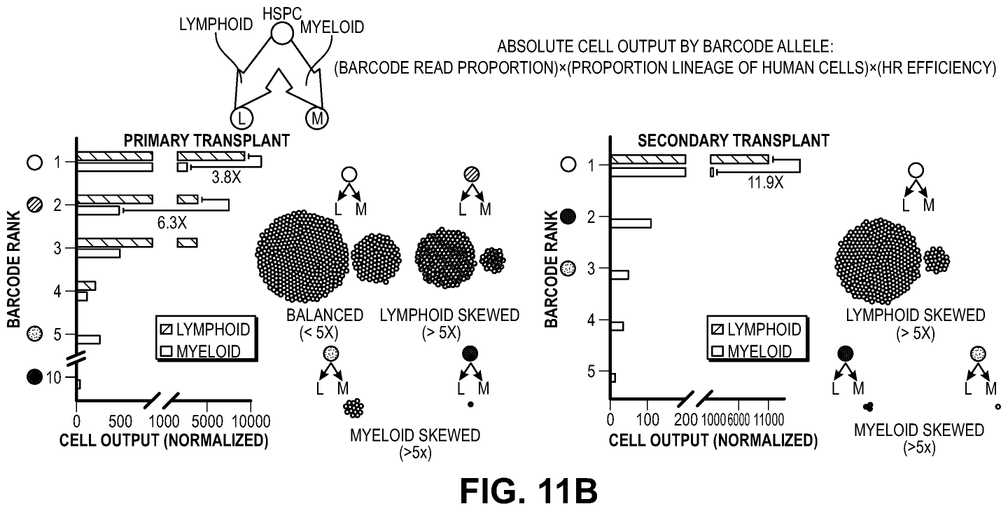

We targeted cord blood-derived HSPCs with the AAVS1-BC pool of AAV6 donor templates and transplanted them into sublethally irradiated NSG mice to assess the clonal contribution via TRACE-Seq. Robust AAVS1-BC donor targeting into the AAVS 1 locus was achieved in two independent experiments across five HSPC donors and a mean of 2.90±0.4×10⁵ cells transplanted per NSG mouse (data not shown). Following 16-18 weeks of hematopoietic reconstitution, we observed 45.4%±14.2 human engraftment, with a gene targeting efficiency of 42.4%±11.4 (data not shown). As with the HBB donors, the majority of differentiated cells were CD19⁺ lymphoid and CD33⁺ myeloid cells, with a strong trend towards more genome editing within the CD33⁺ population (55.8±12.0 vs. 22.3±11.2; p=0.06, two-tailed t-test) (data not shown). To assess clonal contributions of AAVS1 targeted HSPCs, lineage specific cells (CD19⁺ or CD33⁺) were sorted (data not shown), and AAVS1-BFP specific amplicons were generated for NGS sequencing of cells with on-target integrations of SFFV-BFP-PolyA. Consistent with our findings targeting the HBB locus, we identified not only similar numbers of unique barcodes (representing individual clones) in divergent hematopoietic lineages (FIG. 11a), but also similar patterns between primary and secondary transplants, suggesting again that TRACE-Seq identifies Cas9/sgRNA and AAV6-mediated targeting of LT-HSCs (FIG. 11a). Across all mice, bi-lineage clones were seen in four out of five mice, with the exception being mouse 38, from which we were not able to sort sufficient numbers of myeloid cells for valid analysis (data not shown). As with HBB TRACE-Seq, calculating the relative cell output of individual barcodes revealed lymphoid skewed, myeloid skewed and balanced HSPC clones (FIG. 11b, left). The most dominant clone (red), which displayed high proliferative output with a more balanced hematopoietic lineage distribution in the primary mouse, was the predominant clone in the secondary transplant (FIG. 11b, right). In addition, we observed less abundant, myeloid skewed clones (blue and green) in both primary and secondary transplants. These results confirm that gene targeted LT-HSC clones contribute to robust multi-lineage engraftment.

### Discussion

TRACE-Seq improves the understanding of the clonal dynamics of hematopoietic stem and progenitor cells following homologous recombination-based genome editing using two different gene targets (HBB and AAVS1). The data demonstrate that Cas9/sgRNA and AAV6 gene editing targets four distinct types of hematopoietic cells capable of engraftment, including: 1) rare and potent hematopoietic balanced LT-HSCs, 2) rare lymphoid skewed progenitors, 3) rare and potent myeloid skewed progenitors, and 4) more common and less proliferative myeloid skewed HSPCs.

TRACE-seq clearly demonstrates that in the NSG mouse model, engraftment of human cells after genome editing is largely oligoclonal with a few clones contributing to the bulk of hematopoiesis. From a technical perspective, we have developed a data analysis pipeline with multiple filters to distinguish sequencing artifacts from low abundance clones. As sequencing technologies and barcode design improve, the ability to distinguish noise from low abundance clones will similarly improve. Nonetheless, the evidence that clones that were seemingly rare in primary transplants can contribute significantly to hematopoiesis in secondary transplants demonstrates both the sensitivity of this method to detect such clones and the biologic importance of such clones in hematopoiesis.

We compare and contrast these results to lentiviral based genetic engineering of HSPCs since clonal dynamics of genome edited cells has not been published previously. Previous studies tracking LV IS in NSG mice have suggested on the order of 10-200 total clones (without data regarding the relative contributions of different clones) persisting long-term (although at different frequencies in each of the two mice analyzed), with identification of lineage-skewed as well as multi-potent LT-HSCs [Cheung, A. M. et al., Blood 122, 3129-3137 (2013)]. Accordingly, TRACE-Seq identified>50 clones per mouse that were contributing to the entire hematopoiesis of gene targeted cells (FIG. 9e), suggesting that genome edited human HSPCs engraft as efficiently as lentiviral engineered cells in the NSG xenogeneic model. Interestingly, we identified 1-3 clones capable of robust multi-lineage reconstitution in secondary transplants, suggesting between one in 6×10⁴ and 4.6×10⁵ input cells are gene targeted LT-HSCs (based on the numbers of cells transplanted). In a clinical trial for Wiskott-Aldrich syndrome (WAS), IS analysis showed the frequency of CD34⁺ HSPCs with steady-state long term lineage reconstitutions falls between 1 in 100,000 and 1 in a 1,000,000 (a few thousand clones out of the ˜80-200 million HSPCs transplanted) [Biasco, L. et al., Cell Stem Cell 19, 107-119 (2016)]. Further building on this clinical trial, recent reports have suggested that LV integrations occur in cells within the HSPC pool that have long-term lymphoid or myeloid lineage restrictions as well [Scala, S. et al., Nat Med 24, 1683-1690 (2018)]. Taken together, our data suggest that the frequency of gene-targeting and LV gene addition are similar in potent long-term engrafting LT-HSCs.

TRACE-Seq also demonstrated genome edited clones that were heavily lineage skewed in both primary and secondary transplants. This finding demonstrates that the gold standard of HSC function, namely serial transplantation, may not always identify multi-potent HSCs. Nonetheless, the method should allow assessment of other mouse xenograft models of human hematopoietic transplantation in supporting lineage restricted and multi-lineage reconstitution of genome edited cells, including models that further maintain healthy and leukemic myeloid and innate immune system development [Reinisch, A. et al., Nat Med 22, 812-821 (2016); Rongvaux, A. et al., Nature biotechnology 35, 1211 (2017); Wunderlich, M. et al., Leukemia 24, 1785-1788 (2010)]. In the future, this method, potentially combined with novel cell sorting schemes to resolve lineage preference within the CD34+ fraction [Notta, F. et al., Science 351, aab2116 (2016)], should help determine whether cells that undergo gene targeting have a bias towards particular lineages which may help guide which human genetic diseases of the blood may be most amenable to gene targeting based approaches. For example, if gene targeting preferentially occurs in long-term myeloid progenitors, this would support its use in diseases that require long-term myeloid engraftment of gene targeted cells such as sickle cell disease, chronic granulomatous disease, or beta-thalassemia.

In addition to helping understand hematopoietic reconstitution of genome edited cells in pre-clinical models, TRACE-Seq could also be used to further investigate the wide variety of genome editing approaches and HSC culture conditions to determine if they change either the degree of polyclonality or the lineage restriction of clones following engraftment. The wide numbers of variables that are under active study include different genome editing reagents and methods (different nucleases and donor templates and the inhibition of certain pathways [Canny, M. D. et al., Nature biotechnology 36, 95-102 (2018); Schiroli, G. et al., Cell Stem Cell 24, 551-565 e558 (2019)]), differing culture conditions (e.g. cytokine variations [Wilkinson, A. C. et al., Nature 571, E12 (2019)], small molecules [Fares, I. et al., Science 345, 1509-1512 (2014); Cohen, S. et al., Lancet Haematol (2019)], peptides [Canny, M. D. et al., Nature biotechnology 36, 95-102 (2018); Schiroli, G. et al., Cell Stem Cell 24, 551-565 e558 (2019)], and 3-D hydrogel scaffolds [Bai, T. et al. Expansion of primitive human hematopoietic stem cells by culture in a zwitterionic hydrogel. Nat Med (2019)]), and altering the metabolic or cell cycle properties of the gene edited cells. This study, in which two different approaches targeting two different genes was established, serves as the key foundation for such future studies.

In conclusion, TRACE-seq demonstrates that homologous recombination-based genome editing can occur in human hematopoietic stem cells as defined by multi-lineage reconstitution following serial transplantation at a single cell, clonal level. Moreover, TRACE-Seq lays the foundation of clonal tracking of gene targeted HSPCs for basic research into normal and malignant hematopoiesis. The ability of track clones in a clinical setting has proven to be a powerful approach to understand the safety, efficacy, and clonal dynamics of lentiviral based gene therapies, and it will be informative to determine if regulatory agencies will accept having innocuous barcodes as part of recombination donor templates in clinical studies so that the safety, efficacy, and clonal dynamics of reconstituted gene targeted cells, including HSCs, T-cells, or other engineered cell types, can be tracked following administration to patients.

### BIBLIOGRAPHY

1. High, K. A. & Roncarolo, M. G. Gene Therapy. N Engl J Med 381, 455-464 (2019).
2. Cavazzana, M., Bushman, F. D., Miccio, A., Andre-Schmutz, I. & Six, E. Gene therapy targeting haematopoietic stem cells for inherited diseases: progress and challenges. Nat Rev Drug Discov 18, 447-462 (2019).
3. Biasco, L. et al. In vivo tracking of T cells in humans unveils decade-long survival and activity of genetically modified T memory stem cells. Sci Transl Med 7, 273ra213 (2015).
4. Biasco, L. et al. In Vivo Tracking of Human Hematopoiesis Reveals Patterns of Clonal Dynamics during Early and Steady-State Reconstitution Phases. Cell Stem Cell 19, 107-119 (2016).
5. Scala, S. et al. Dynamics of genetically engineered hematopoietic stem and progenitor cells after autologous transplantation in humans. Nat Med 24, 1683-1690 (2018).
6. Mamcarz, E. et al. Lentiviral Gene Therapy Combined with Low-Dose Busulfan in Infants with SCID-X1. N Engl J Med 380, 1525-1534 (2019).
7. Fraietta, J. A. et al. Disruption of TET2 promotes the therapeutic efficacy of CD19-targeted T cells. Nature 558, 307-312 (2018).
8. Marktel, S. et al. Intrabone hematopoietic stem cell gene therapy for adult and pediatric patients affected by transfusion-dependent ss-thalassemia. Nat Med 25, 234-241 (2019).
9. Porter, S. N., Baker, L. C., Mittelman, D. & Porteus, M. H. Lentiviral and targeted cellular barcoding reveals ongoing clonal dynamics of cell lines in vitro and in vivo. Genome Biol 15, R75 (2014).
10. Lu, R., Neff, N. F., Quake, S. R. & Weissman, I. L. Tracking single hematopoietic stem cells in vivo using high-throughput sequencing in conjunction with viral genetic barcoding. Nature biotechnology 29, 928-933 (2011).
11. Yabe, I. M. et al. Barcoding of Macaque Hematopoietic Stem and Progenitor Cells: A Robust Platform to Assess Vector Genotoxicity. Mol Ther Methods Clin Dev 11, 143-154 (2018).
12. Wu, C. et al. Clonal tracking of rhesus macaque hematopoiesis highlights a distinct lineage origin for natural killer cells. Cell Stem Cell 14, 486-499 (2014).
13. Kristiansen, T. A. et al. Cellular Barcoding Links B-1a B Cell Potential to a Fetal Hematopoietic Stem Cell State at the Single-Cell Level. Immunity 45, 346-357 (2016).
14. Thielecke, L. et al. Limitations and challenges of genetic barcode quantification. Sci Rep 7, 43249 (2017).
15. Barzel, A. et al. Promoterless gene targeting without nucleases ameliorates haemophilia B in mice. Nature 517, 360-364 (2015).
16. Russell, D. W. & Hirata, R. K. Human gene targeting by viral vectors. Nat Genet 18, 325-330 (1998).
17. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
18. Anzalone, A. V. et al. Search-and-replace genome editing without double-strand breaks or donor DNA. Nature (2019).
19. Miller, D. G., Petek, L. M. & Russell, D. W. Human gene targeting by adeno-associated virus vectors is enhanced by DNA double-strand breaks. Mol Cell Biol 23, 3550-3557 (2003).
20. Porteus, M. H. & Baltimore, D. Chimeric nucleases stimulate gene targeting in human cells. Science 300, 763 (2003).
21. Genovese, P. et al. Targeted genome editing in human repopulating haematopoietic stem cells. Nature 510, 235-240 (2014).
22. Urnov, F. D. et al. Highly efficient endogenous human gene correction using designed zinc-finger nucleases. Nature 435, 646-651 (2005).
23. Porteus, M. H. & Carroll, D. Gene targeting using zinc finger nucleases. Nature biotechnology 23, 967-973 (2005).
24. Lombardo, A. et al. Site-specific integration and tailoring of cassette design for sustainable gene transfer. Nat Methods 8, 861-869 (2011).
25. Jinek, M. et al. A programmable dual-RNA-guided DNA endonuclease in adaptive bacterial immunity. Science 337, 816-821 (2012).
26. Cong, L. et al. Multiplex genome engineering using CRISPR/Cas systems. Science 339, 819-823 (2013).
27. Vakulskas, C. A. et al. A high-fidelity Cas9 mutant delivered as a ribonucleoprotein complex enables efficient gene editing in human hematopoietic stem and progenitor cells. Nat Med 24, 1216-1224 (2018).
28. Porteus, M. H. A New Class of Medicines through DNA Editing. N Engl J Med 380, 947-959 (2019).
29. Martin, R. M. et al. Highly Efficient and Marker-free Genome Editing of Human Pluripotent Stem Cells by CRISPR-Cas9 RNP and AAV6 Donor-Mediated Homologous Recombination. Cell Stem Cell 24, 821-828 e825 (2019).
30. Dever, D. P. et al. CRISPR/Cas9 beta-globin gene targeting in human haematopoietic stem cells. Nature 539, 384-389 (2016).
31. Pavel-Dinu, M. et al. Gene correction for SCID-X1 in long-term hematopoietic stem cells. Nat Commun 10, 1634 (2019).
32. Schiroli, G. et al. Preclinical modeling highlights the therapeutic potential of hematopoietic stem cell gene editing for correction of SCID-X1. Sci Transl Med 9 (2017).
33. Gomez-Ospina, N. et al. Human genome-edited hematopoietic stem cells phenotypically correct Mucopolysaccharidosis type I. Nat Commun 10, 4045 (2019).
34. De Ravin, S. S. et al. CRISPR-Cas9 gene repair of hematopoietic stem cells from patients with X-linked chronic granulomatous disease. Sci Transl Med 9 (2017).
35. Hubbard, N. et al. Targeted gene editing restores regulated CD40L function in X-linked hyper-IgM syndrome. Blood 127, 2513-2522 (2016).
36. Eyquem, J. et al. Targeting a CAR to the TRAC locus with CRISPR/Cas9 enhances tumour rejection. Nature 543, 113-117 (2017).
37. van Overbeek, M. et al. DNA Repair Profiling Reveals Nonrandom Outcomes at Cas9-Mediated Breaks. Mol Cell 63, 633-646 (2016).
38. Davidsson, M. et al. A novel process of viral vector barcoding and library preparation enables high-diversity library generation and recombination-free paired-end sequencing. Sci Rep 6, 37563 (2016).
39. Bak, R. O., Dever, D. P. & Porteus, M. H. CRISPR/Cas9 genome editing in human hematopoietic stem cells. Nat Protoc 13, 358-376 (2018).
40. Aurnhammer, C. et al. Universal real-time PCR for the detection and quantification of adeno-associated virus serotype 2-derived inverted terminal repeat sequences. Hum Gene Ther Methods 23, 18-28 (2012).
41. Hendel, A. et al. Chemically modified guide RNAs enhance CRISPR-Cas genome editing in human primary cells. Nature biotechnology 33, 985-989 (2015).
42. Dulmovits, B. M. et al. Pomalidomide reverses gamma-globin silencing through the transcriptional reprogramming of adult hematopoietic progenitors. Blood 127, 1481-1492 (2016).
43. Hu, J. et al. Isolation and functional characterization of human erythroblasts at distinct stages: implications for understanding of normal and disordered erythropoiesis in vivo. Blood 121, 3246-3253 (2013).
44. Costello, M. et al. Characterization and remediation of sample index swaps by non-redundant dual indexing on massively parallel sequencing platforms. BMC Genomics 19, 332 (2018).
45. Sinha, R. et al. Index switching causes “spreading-of-signal” among multiplexed samples in Illumina HiSeq 4000 DNA sequencing. bioRxiv, 125724 (2017).
46. Larsson, A. J. M., Stanley, G., Sinha, R., Weissman, I. L. & Sandberg, R. Computational correction of index switching in multiplexed sequencing libraries. Nat Methods 15, 305-307 (2018).
47. Rogers, Z. N. et al. A quantitative and multiplexed approach to uncover the fitness landscape of tumor suppression in vivo. Nat Methods 14, 737-742 (2017).
48. Zhang, J., Kobert, K., Flouri, T. & Stamatakis, A. PEAR: a fast and accurate Illumina Paired-End reAd mergeR. Bioinformatics 30, 614-620 (2014).
49. Doulatov, S., Notta, F., Laurenti, E. & Dick, J. E. Hematopoiesis: a human perspective. Cell Stem Cell 10, 120-136 (2012).
50. Cheung, A. M. et al. Analysis of the clonal growth and differentiation dynamics of primitive barcoded human cord blood cells in NSG mice. Blood 122, 3129-3137 (2013).
51. Reinisch, A. et al. A humanized bone marrow ossicle xenotransplantation model enables improved engraftment of healthy and leukemic human hematopoietic cells. Nat Med 22, 812-821 (2016).
52. Rongvaux, A. et al. Corrigendum: Development and function of human innate immune cells in a humanized mouse model. Nature biotechnology 35, 1211 (2017).
53. Wunderlich, M. et al. AML xenograft efficiency is significantly improved in NOD/SCID-IL2RG mice constitutively expressing human SCF, GM-CSF and IL-3. Leukemia 24, 1785-1788 (2010).
54. Notta, F. et al. Distinct routes of lineage development reshape the human blood hierarchy across ontogeny. Science 351, aab2116 (2016).
55. Canny, M. D. et al. Inhibition of 53BP1 favors homology-dependent DNA repair and increases CRISPR-Cas9 genome-editing efficiency. Nature biotechnology 36, 95-102 (2018).
56. Schiroli, G. et al. Precise Gene Editing Preserves Hematopoietic Stem Cell Function following Transient p53-Mediated DNA Damage Response. Cell Stem Cell 24, 551-565 e558 (2019).
57. Wilkinson, A. C. et al. Author Correction: Long-term ex vivo haematopoietic-stem-cell expansion allows nonconditioned transplantation. Nature 571, E12 (2019).
58. Fares, I. et al. Cord blood expansion. Pyrimidoindole derivatives are agonists of human hematopoietic stem cell self-renewal. Science 345, 1509-1512 (2014).
59. Cohen, S. et al. Hematopoietic stem cell transplantation using single UM171-expanded cord blood: a single-arm, phase 1-2 safety and feasibility study. Lancet Haematol (2019).
60. Bai, T. et al. Expansion of primitive human hematopoietic stem cells by culture in a zwitterionic hydrogel. Nat Med (2019).

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. All publications, patents, and patent applications cited in this specification are herein incorporated by reference as if each individual publication, patent, or patent application were specifically and individually indicated to be incorporated by reference.

1. A method of tracking cell populations comprising an introduced DNA molecule, the method comprising
introducing a plurality of homology recombination donor template polynucleotide sequences into a plurality of cells under conditions such that at least part of the homology recombination donor template polynucleotide sequences are introduced into a target genomic sequence of a cell from the cell population, wherein the homology recombination donor template polynucleotide sequences comprise in the following order: a left homology arm, a coding sequence, and a right homology arm, wherein
(1) the coding sequence comprises a silent mutation compared to a wildtype coding sequence of the cell, wherein the plurality of homology recombination donor template polynucleotide sequences comprises different silent mutations and wherein at least two cells receive recombined polynucleotides, each having a different silent mutation; or
(2) between the left and right homology arms and outside the coding sequence a barcode sequence is present, wherein the plurality comprises different barcodes and wherein at least two cells receive recombined polynucleotides, each having a different barcode sequence.

2. The method of claim 1, wherein the plurality of homology recombination donor template polynucleotide sequences comprises at least 10 different silent mutations and wherein at least 10 cells receive recombined polynucleotides, each having a different silent mutation

3. The method of claim 1, wherein the plurality comprises at least 10 different barcodes and wherein at least 10 cells receive recombined polynucleotides, each having a different barcode sequence.

4. The method of claim 1, wherein between the left and right homology arms and outside the coding sequence the barcode sequence is present and wherein following the coding sequences there is a polyA sequence and the barcode is present between the polyA sequence and the right homology arm.

5. The method of claim 1, wherein the cells are primary cells.

6. The method of claim 5, wherein the cells are primary hematopoietic cells.

7. The method of claim 6, wherein the cells are primary hematopoietic stem cells.

8. The method of claim 6, wherein the cells are primary T-cells.

9. (canceled)

10. The method of claim 1, wherein the introducing comprises providing a targeted nuclease into the cell wherein the targeted nuclease introduces a double-stranded break in the genomic DNA of the cell at a sequence in the genome to which the right and left homology arm sequences have homology.

11. The method of claim 10, wherein the targeted nuclease is targeted by a single guide RNA (sgRNA).

12. The method of claim 11, wherein the sgRNA comprises one or more modified nucleotides.

13. The method of claim 11, wherein the targeted nuclease comprises CRISPR-associated protein (Cas) polypeptide.

14. The method of claim 10, wherein the targeted nuclease comprises a zinc finger nuclease (ZFN), a transcription activator-like effector nuclease (TALEN) or a meganuclease.

15. The method of claim 1, wherein the introducing comprises introducing adeno-associated viral (AAV) vectors comprising the homology recombination donor template polynucleotide sequences.

16. The method of claim 15, wherein the introducing further comprises introducing into the cells a ribonucleoprotein (RNP) comprising a single guide RNA (sgRNA) and a CRISPR-associated protein (Cas) polypeptide.

17. The method of claim 1, further comprising allowing the cell population to divide thereby forming an expanded cell population; and
sequencing recombined polynucleotides from the expanded cell population, thereby allowing for tracking of different cells based on the different silent mutations or different barcodes.

18. The method of claim 17, wherein the cells are primary hematopoietic cells and the allowing comprises introducing the cells into an animal and the cells divide and optionally differentiate in the animal.

19-21. (canceled)

22. The method of claim 1, wherein the coding sequence encodes hemoglobin (HBB), Wiskott-Aldrich Syndrome Protein (WAS), Iduronidase (IDUA), Interleukin-7 receptor alpha (Il7RA), Interleukin-2 receptor gamma chain (Il2RG), gp91phox (CYBB), V(D)J recombination-activating protein 1(RAG), V(D)J recombination-activating protein 2 (RAG2), Galactosylceramidase (GALC), Tripeptidyl-peptidase 1(TPP), Glucosylcermidase beta (GBA), Cystic Fibrosis Transmembrane Receptor (CFTR), Forxhead box protein P3 (FOXP3), CD40 Ligand (CD40L), Perforin 1 (PRF1), T-cell Receptor (TCR), Beta-2-microglobulin (B2M), ATP-binding cassette sub-family D member1 (ABCD-1), Brain-derived neurotrophic factor (BDNF), or phenylalanine hydroxylase (PAH).

23. (canceled)

24. A plurality of homology recombination donor template polynucleotide sequences comprising in the following order: a left homology arm, a coding sequence, and a right homology arm, wherein
(1) the coding sequence comprises a silent mutation compared to a wildtype coding sequence, wherein the plurality comprises at least two different silent mutations; or
(2) between the left and right homology arms and outside the coding sequence a barcode sequence is present, wherein the plurality comprises at least two different barcodes.

25-46. (canceled)


SEQUENCE LISTING (41 sequences)

SEQ ID NO: 1 - DNA, 645 bp, Artificial Sequence
  Description of Artificial Sequence Synthetic polynucleotide
  modified_base (202)..(202): a, c, t, g, unknown or other
  modified_base (211)..(211): a, c, t, g, unknown or other
  modified_base (214)..(214): a, c, t, g, unknown or other
  modified_base (226)..(226): a, c, t, g, unknown or other
agaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtncayttracnccngargaraartcngcagtcactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtga

SEQ ID NO: 2 - DNA, 645 bp, Artificial Sequence
  Description of Artificial Sequence Synthetic polynucleotide
  modified_base (202)..(202): a, c, t, g, unknown or other
  modified_base (211)..(211): a, c, t, g, unknown or other
  modified_base (214)..(214): a, c, t, g, unknown or other
agaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtncayttracnccngargaraaragygcagtcactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtga

SEQ ID NO: 3 - DNA, 645 bp, Artificial Sequence
  Description of Artificial Sequence Synthetic polynucleotide
  modified_base (202)..(202): a, c, t, g, unknown or other
  modified_base (208)..(208): a, c, t, g, unknown or other
  modified_base (211)..(211): a, c, t, g, unknown or other
  modified_base (214)..(214): a, c, t, g, unknown or other
  modified_base (226)..(226): a, c, t, g, unknown or other
agaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtncayctnacnccngargaraartcngcagtcactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtga

SEQ ID NO: 4 - DNA, 645 bp, Artificial Sequence
  Description of Artificial Sequence Synthetic polynucleotide
  modified_base (202)..(202): a, c, t, g, unknown or other
  modified_base (208)..(208): a, c, t, g, unknown or other
  modified_base (211)..(211): a, c, t, g, unknown or other
  modified_base (214)..(214): a, c, t, g, unknown or other
agaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtncayctnacnccngargaraaragygcagtcactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtga

SEQ ID NO: 5 - DNA, 12 bp, Artificial Sequence
  Description of Artificial Sequence Synthetic oligonucleotide
vhdbvhdbvhdb

SEQ ID NO: 6 - DNA, 38 bp, Artificial Sequence
  Description of Artificial Sequence Synthetic primer
ccatcactaggggttcctgcggccgccaccgtttttct

SEQ ID NO: 7 - DNA, 30 bp, Artificial Sequence
  Description of Artificial Sequence Synthetic primer
ttaattaagcttgtgccccagtttgctagg

SEQ ID NO: 8 - DNA, 43 bp, Artificial Sequence
  Description of Artificial Sequence Synthetic primer
tggggcacaagcttaattaavhdbvhdbvhdbctcgagggcgc

SEQ ID NO: 9 - DNA, 38 bp, Artificial Sequence
  Description of Artificial Sequence Synthetic primer
ccatcactaggggttcctgcggccgcagaactcaggac

SEQ ID NO: 10 - DNA, 20 bp, Artificial Sequence
  Description of Artificial Sequence Synthetic oligonucleotide
cttgccccacagggcagtaa

SEQ ID NO: 11 - DNA, 20 bp, Artificial Sequence
  Description of Artificial Sequence Synthetic oligonucleotide
ggggccactagggacaggat

SEQ ID NO: 12 - protein, 9 aa, Homo sapiens
VHLTPEEKS

SEQ ID NO: 13 - DNA, 93 bp, Homo sapiens
caaacagacaccatggtgcacctgactcctgaggaaaaatccgcagtcactgccctgtggggcaaggtgaacgtggatgaagttggtggtgag

SEQ ID NO: 14 - protein, 27 aa, Homo sapiens
MVHLTPEEKSAVTALWGKVNVDEVGGE

SEQ ID NO: 15 - protein, 12 aa, Homo sapiens
VHLTPEEKSAVT

SEQ ID NO: 16 - DNA, 66 bp, Artificial Sequence
  Description of Artificial Sequence Synthetic oligonucleotide
  modified_base (1)..(7): a, c, t, g, unknown or other
  modified_base (41)..(66): a, c, t, g, unknown or other
nnnnnnntggtgcacttgacccctgaggagaartccgcagnnnnnnnnnnnnnnnnnnnnnnnnnn

SEQ ID NO: 17 - protein, 22 aa, Artificial Sequence
  Description of Artificial Sequence Synthetic peptide
  MOD_RES (1)..(3): Any amino acid
  MOD_RES (14)..(22): Any amino acid
XXXVHLTPEEKSAXXXXXXXXX

SEQ ID NO: 18 - DNA, 66 bp, Homo sapiens
gacaccatggtgcatctgactcctgaggagaagtctgccgttactgccctgtggggcaaggtgaac

SEQ ID NO: 19 - protein, 22 aa, Homo sapiens
DTMVHLTPEEKSAVTALWGKVN

SEQ ID NO: 20 - DNA, 33 bp, Artificial Sequence
  Description of Artificial Sequence Synthetic oligonucleotide
tggtccacctaacccctgaagagaagagtgcag

SEQ ID NO: 21 - protein, 10 aa, Artificial Sequence
  Description of Artificial Sequence Synthetic peptide
VHLTPEEKSA

SEQ ID NO: 22 - DNA, 33 bp, Artificial Sequence
  Description of Artificial Sequence Synthetic oligonucleotide
tggtacacttaacgcctgaagagaaatctgcag

SEQ ID NO: 23 - DNA, 33 bp, Artificial Sequence
  Description of Artificial Sequence Synthetic oligonucleotide
tggtgcacctgactcctgaggaaaaatccgcag

SEQ ID NO: 24 - DNA, 33 bp, Artificial Sequence
  Description of Artificial Sequence Synthetic oligonucleotide
tggttcacttaactcctgaggagaagtccgcag

SEQ ID NO: 25 - DNA, 33 bp, Artificial Sequence
  Description of Artificial Sequence Synthetic oligonucleotide
tggttcacttaacaccagaagagaaatctgcag

SEQ ID NO: 26 - DNA, 33 bp, Artificial Sequence
  Description of Artificial Sequence Synthetic oligonucleotide
tggttcatttgaccccggaggagaagtcggcag

SEQ ID NO: 27 - DNA, 33 bp, Artificial Sequence
  Description of Artificial Sequence Synthetic oligonucleotide
tggtacacttgacccctgaggaaaagtcggcag

SEQ ID NO: 28 - DNA, 33 bp, Artificial Sequence
  Description of Artificial Sequence Synthetic oligonucleotide
tggttcacttgacaccggaggagaaatcggcag

SEQ ID NO: 29 - DNA, 33 bp, Artificial Sequence
  Description of Artificial Sequence Synthetic oligonucleotide
tggtgcatctcactccggaagagaagagcgcag

SEQ ID NO: 30 - DNA, 33 bp, Artificial Sequence
  Description of Artificial Sequence Synthetic oligonucleotide
tggtgcatcttacacctgaggaaaaaagcgcag

SEQ ID NO: 31 - DNA, 33 bp, Artificial Sequence
  Description of Artificial Sequence Synthetic oligonucleotide
tggtccatttgaccccggaggagaaatctgcag

SEQ ID NO: 32 - DNA, 33 bp, Artificial Sequence
  Description of Artificial Sequence Synthetic oligonucleotide
tggtgcatctgacgccagaggagaagagcgcag

SEQ ID NO: 33 - DNA, 33 bp, Artificial Sequence
  Description of Artificial Sequence Synthetic oligonucleotide
tggtgcacttaacacctgaagagaagtcggcag

SEQ ID NO: 34 - DNA, 33 bp, Artificial Sequence
  Description of Artificial Sequence Synthetic oligonucleotide
tggtccatcttacccccgaagagaaaagcgcag

SEQ ID NO: 35 - DNA, 72 bp, Homo sapiens
caacctcaaacagacaccatggtgcacctgactcctgaggagaagtctgccgttactgccctgtggggcaag

SEQ ID NO: 36 - DNA, 72 bp, Artificial Sequence
  Description of Artificial Sequence Synthetic oligonucleotide
  modified_base (24)..(24): a, c, t, g, unknown or other
  modified_base (27)..(28): a, c, t, g, unknown or other
  modified_base (30)..(30): a, c, t, g, unknown or other
  modified_base (33)..(33): a, c, t, g, unknown or other
  modified_base (36)..(36): a, c, t, g, unknown or other
  modified_base (39)..(39): a, c, t, g, unknown or other
  modified_base (42)..(42): a, c, t, g, unknown or other
  modified_base (45)..(48): a, c, t, g, unknown or other
caacctcaaacagacaccatggtncanntnacnccnganganaannnngcagtcactgccctgtggggcaag

SEQ ID NO: 37 - protein, 11 aa, Homo sapiens
MVHLTPEEKSA

SEQ ID NO: 38 - DNA, 37 bp, Artificial Sequence
  Description of Artificial Sequence Synthetic oligonucleotide
  modified_base (6)..(6): a, c, t, g, unknown or other
  modified_base (15)..(15): a, c, t, g, unknown or other
  modified_base (18)..(18): a, c, t, g, unknown or other
  modified_base (30)..(30): a, c, t, g, unknown or other
atggtncayttracnccngargaraartcngcagtcc

SEQ ID NO: 39 - DNA, 37 bp, Artificial Sequence
  Description of Artificial Sequence Synthetic oligonucleotide
  modified_base (6)..(6): a, c, t, g, unknown or other
  modified_base (15)..(15): a, c, t, g, unknown or other
  modified_base (18)..(18): a, c, t, g, unknown or other
atggtncayttracnccngargaraaragygcagtcc

SEQ ID NO: 40 - DNA, 37 bp, Artificial Sequence
  Description of Artificial Sequence Synthetic oligonucleotide
  modified_base (6)..(6): a, c, t, g, unknown or other
  modified_base (12)..(12): a, c, t, g, unknown or other
  modified_base (15)..(15): a, c, t, g, unknown or other
  modified_base (18)..(18): a, c, t, g, unknown or other
  modified_base (30)..(30): a, c, t, g, unknown or other
atggtncayctnacnccngargaraartcngcagtcc

SEQ ID NO: 41 - DNA, 37 bp, Artificial Sequence
  Description of Artificial Sequence Synthetic oligonucleotide
  modified_base (6)..(6): a, c, t, g, unknown or other
  modified_base (12)..(12): a, c, t, g, unknown or other
  modified_base (15)..(15): a, c, t, g, unknown or other
  modified_base (18)..(18): a, c, t, g, unknown or other
atggtncayctnacnccngargaraaragygcagtcc